(19) United States
(12) Patent Application Publication
(10) Pub. No.: US 2011/0015392 A1
(43) Pub. Date: Jan. 20, 2011

(54) PROCESS FOR PREPARING PYRIDONE DERIVATIVES 226

(75) Inventors: Mark Richard Fielding, Macclesfield (GB); John Leonard, Macclesfield (GB)

Correspondence Address: ASTRAZENECA R&D BOSTON, 35 GATEHOUSE DRIVE, WALTHAM, MA, 02451-1215, US

(73) Assignee: ASTRAZENECA AB

(21) Appl. No.: 12/854,388

(22) Filed: Aug. 11, 2010

Related U.S. Application Data
(63) Continuation of application No. PCT/GB2009/050124, filed on Feb. 9, 2009.
(60) Provisional application No. 61/027,582, filed on Feb. 11, 2008.

(51) Int. Cl. C07D 265/32; C07D 213/80

(52) U.S. Cl. 544/98; 546/297; 546/250

(57) ABSTRACT

A route for preparing a compound of formula (I)

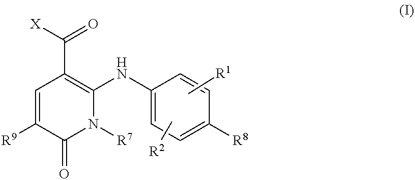

where X, R¹, R², R⁷, R⁸ and R⁹ are as defined in the specification, is described. Process steps used in the route and novel intermediates prepared during the route are also described and claimed. Compounds of formula (I) are used in the preparation of pharmaceutical compounds, in particular inhibitors of MEK, useful in the treatment of hyperproliferative disease such as cancer and inflammatory conditions.

This is a Continuation Application of International Application No. PCT/GB2009/050124 (filed Feb. 9, 2009), which claims priority to U.S. Provisional Application No. 61/027,582 filed on Feb. 11, 2008.

The present invention relates to a novel route for preparing compounds useful in the synthesis of pharmaceutically active compounds, as well as novel processes and intermediates and compounds used in the route.

Cell signalling through growth factor receptors and protein kinases is an important regulator of cell growth, proliferation and differentiation. In normal cell growth, growth factors, through receptor activation (i.e. PDGF or EGF and others), activate MAP kinase pathways. One of the most important and most well understood MAP kinase pathways involved in normal and uncontrolled cell growth is the Ras/Raf kinase pathway. Active GTP-bound Ras results in the activation and indirect phosphorylation of Raf kinase. Raf is then phosphorylates MEK1 and 2 on two serine residues (S218 and S222 for MEK1 and S222 and S226 for MEK2) (Ahn et al., Methods in Enzymology, 2001, 332, 417-431). Activated MEK then phosphorylates its only known substrates, the MAP kinases, ERK1 and 2. ERK phosphorylation by MEK occurs on Y204 and T202 for ERK1 and Y1 85 and T 183 for ERK2 (Ahn et al., Methods in Enzymology, 2001, 332, 417-431). Phosphorylated ERK dimerizes and then translocates to the nucleus where it accumulates (Khokhlatchev et al., Cell, 1998, 93, 605-615). In the nucleus, ERK is involved in several important cellular functions, including but not limited to nuclear transport, signal transduction, DNA repair, nucleosome assembly and translocation, and mRNA processing and translation (Ahn et al., Molecular Cell, 2000, 6, 1343-1354). Overall, treatment of cells with growth factors leads to the activation of ERK1 and 2 which results in proliferation and, in some cases, differentiation (Lewis et al., Adv. Cancer Res., 1998, 74, 49-139).

In proliferative diseases, genetic mutations and/or overexpression of the growth factor receptors, downstream signalling proteins, or protein kinases involved in the ERK kinase pathway lead to uncontrolled cell proliferation and, eventually, tumor formation. For example, some cancers contain mutations which result in the continuous activation of this pathway due to continuous production of growth factors. Other mutations can lead to defects in the deactivation of the activated GTP-bound Ras complex, again resulting in activation of the MAP kinase pathway. Mutated, oncogenic forms of Ras are found in 50% of colon and >90% pancreatic cancers as well as many others types of cancers (Kohl et al., Science, 1993, 260, 1834-1837). Recently, bRaf mutations have been identified in more than 60% of malignant melanoma (Davies, H. et al., Nature, 2002, 417, 949-954). These mutations in bRaf result in a constitutively active MAP kinase cascade. Studies of primary tumor samples and cell lines have also shown constitutive or overactivation of the MAP kinase pathway in cancers of pancreas, colon, lung, ovary and kidney (Hoshino, R. et al., Oncogene, 1999, 18, 813-822). Hence, there is a strong correlation between cancers and an overactive MAP kinase pathway resulting from genetic mutations.

As constitutive or overactivation of MAP kinase cascade plays a pivotal role in cell proliferation and differentiation, inhibition of this pathway is believed to be beneficial in hyperproliferative diseases. MEK is a key player in this pathway as it is downstream of Ras and Raf. Additionally, it is an attractive therapeutic target because the only known substrates for MEK phosphorylation are the MAP kinases, ERK1 and 2. Inhibition of MEK has been shown to have potential therapeutic benefit in several studies. For example, small molecule MEK inhibitors have been shown to inhibit human tumor growth in nude mouse xenografts, (Sebolt-Leopold et al., Nature-Medicine, 1999, 5 (7), 810-816; Trachet et al., AACR Apr. 6-10, 2002, Poster #5426; Tecle, H., IBC 2nd International Conference of Protein Kinases, Sep. 9-10, 2002), block static allodynia in animals (WO 01/05390) and inhibit growth of acute myeloid leukemia cells (Milella et al., J. CHn. Invest., 2001, 108 (6), 851-859).

Small molecule inhibitors of MEK have been disclosed in a wide range of publications including in U.S. Patent Publication Nos. 2003/0232869, 2004/0116710, and 2003/0216460, and U.S. patent application Ser. Nos. 10/654,580 and 10/929,295, U.S. Pat. No. 5,525,625; WO 98/43960; WO 99/01421; WO 99/01426; WO 00/41505; WO 00/42002; WO 00/42003; WO 00/41994; WO 00/42022; WO 00/42029; WO 00/68201; WO 01/68619; WO 02/06213; WO 03/077914; WO 03/077855, WO2005/051906, WO2005/023759 and WO2005/051301.

A range of heterocyclic compounds, and pharmaceutically acceptable salts and prodrugs thereof, which are potent inhibitors of the MEK enzyme and so are useful in the treatment of hyperproliferative diseases are described in WO2005/051301 and WO2007/044084. In particular, 6-oxo-1,6-dihydropyridine compounds are described in these references, and specifically those of formula A are described and claimed in WO2007/044084:

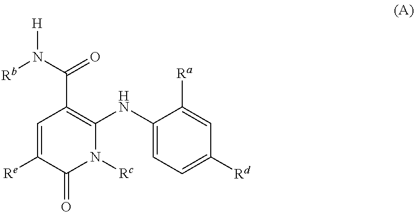

where R^{a} is Cl or F,
R^{b} is hydrogen, methyl, ethyl, hydroxy, methoxy, ethoxy, HO—CH₂—CH₂—O—, HOCH₂C(CH₃)₂)—, (S)—H₃CCH(OH)CH₂O—, (R)—HOCH₂CH(OH)CH₂O—, cyclopropyl-CH₂₋O—, HOCH₂CH₂—,

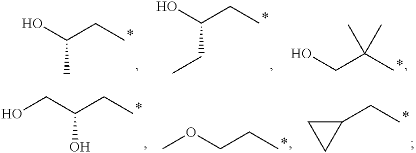

R^{c} is methyl or ethyl, where said methyl and ethyl are optionally substituted with one or more fluorine atoms,

### R^{d} is Br, I or SCH₃,

R^{e} is H, CN, Cl or C₁₋₄alkyl optionally substituted by one or more groups independently selected from F or CN, subject to the exclusion of certain combinations of variables as is described.

The preparation of compounds of formula (A) is also described and claimed in this reference. Specifically, the compounds of formula (A) are prepared by amidation of a compound of formula (B), using a lithiated amine formed using lithium hexamethyldisilazide as the lithiating reagent

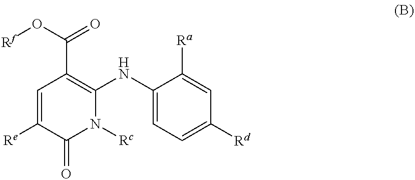

where R^{a}, R^{c}, R^{d}, R^{e} are as defined above, and R^{f} is an alkyl group such as methyl or ethyl.

Compounds of formula (B) are therefore key intermediates in the preparation of the pharmaceutical compounds. They in themselves are generally prepared by reacting a suitable pyridone with the lithiated form of an appropriately substituted aniline derivative, which is generated using lithium hexamethyl disilazide as a base at low temperature. Pyridones required in the process are of formula (C)

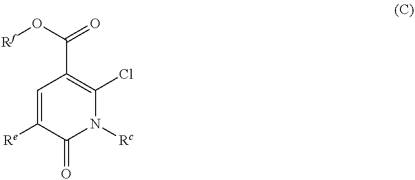

According to US2007/0112038 these pyridones are prepared in 4 synthetic stages starting from 2,6-dichloronicotinic acid. The overall route to the drug from this starting material was 7 stages and was adequate for manufacture of small quantities of material for early evaluation of medicinal properties. However, there are a number of issues that compromise the effectiveness of this route for the large-scale manufacture of the drug.

Included in the problems associated with this route are the following issues: several of the steps produce isomer mixtures that are difficult to separate without chromatography; R^{e} is introduced by a palladium catalysed alkylation step, which is low yielding, does not proceed to completion and leaves residual palladium that is difficult to remove; a number of the stages require low temperatures that are difficult to achieve in manufacturing facilities; the supply of lithium hexamethyl disilazide, which is used for two of the stages, is presently limited and the residue is a potential environmental hazard; and the reagent used to introduce the preferred side chain R^{f}═NHO(CH₂)₂OH(O-(2-vinyloxy-ethyl)-hydroxylamine) is hazardous to prepare and purify.

There is therefore a need for an alternative process for the preparation of pyridones of formula (B), in particular compounds related to compounds of formula A, where R^{c} and R^{e} are methyl groups, but also analogues thereof.

The applicants have developed a new route to 6-oxo-1,6-dihydropyridine compounds such as those of formula (A) and analogues thereof which avoids the difficulties encountered in other routes, as described above. The route is very atom efficient, uses very simple, cheap and readily available reagents, and all steps can be carried out safely at moderate temperatures. The approach is extremely practical and efficient and suitable for the manufacture of these compounds on a commercial scale.

According to a first aspect of the invention, there is provided a method for preparing a compound of formula (I)

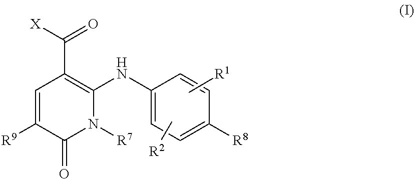

or a pharmaceutically acceptable salt thereof;
where:
R⁷ is methyl or ethyl, either of which are optionally substituted with one or more fluorine atoms;
R¹, R², R⁸ and R⁹ are independently hydrogen, hydroxy, halogen, cyano, nitro, trifluoromethyl, difluoromethyl, fluoromethyl, fluoromethoxy, difluoromethoxy, trifluoromethoxy, azido, —SR²¹, —OR²³, —C(O)R²³, —C(O)OR²³, NR²⁴C(O)OR²⁶, —OC(O)R²³, —NR²⁴SO₂R²⁶, —SO₂NR²³R²⁴, —NR²⁴C(O)R²³, —C(O)NR²³R²⁴, —NR²⁵C(O)NR²³R²⁴, —NR²⁵C(NCN)NR²³R²⁴, —NR²³R²⁴, C₁₋₁₀ alkyl, C₂₋₁₀ alkenyl, C₂₋₁₀alkynyl, C₃₋₁₀cycloalkyl, C₃₋₁₀cycloalkylalkyl, —S(O)ⱼC₁₋₆alkyl, —S(O)ⱼ(CR²⁴R²⁵)ₘ-aryl, aryl, arylalkyl, heteroaryl, heteroarylalkyl, heterocyclyl, heterocyclylalkyl, —O(CR²⁴R²⁵)ₘaryl, —NR²⁴(CR²⁴R²⁵)ₘ-aryl, —O(CR²⁴R²⁵)ₘ-heteroaryl, —NR²⁴(CR²⁴R²⁵)ₘ-heteroaryl, —O(CR²⁴R²⁵)ₘ-heterocyclyl or —NR²⁴(CR²⁴R²⁵)ₘ-heterocyclyl, wherein any of said alkyl, alkenyl, alkynyl, cycloalkyl, aryl, arylalkyl, heteroaryl, heteroarylalkyl, heterocyclyl and heterocyclylalkyl portions are optionally substituted with one or more groups independently selected from oxo (with the proviso that it is not substituted on an aryl or heteroaryl), halogen, cyano, nitro, trifluoromethyl, difluoromethoxy, trifluoromethoxy, azido, —NR²⁴SO₂R²⁶, —SO₂NR²³R²⁴, —C(O)R²³, —C(O)OR²³, —OC(O)R²³, —NR²⁴C(O)OR²⁶, —NR²⁴C(O)R²³, —C(O)NR²³R²⁴, —NR²³R²⁴, —NR²⁵C(O)NR²³R²⁴, —NR²⁵C(NCN)NR²³R²⁴, —OR²³, aryl, heteroaryl, arylalkyl, heteroarylalkyl, heterocyclyl, and heterocyclylalkyl, and wherein said aryl, heteroaryl, arylalkyl, heteroarylalkyl, heterocyclyl or heterocyclylalkyl rings may be further substituted with one or more groups selected from halogen, hydroxyl, cyano, nitro, azido, fluoromethyl, difluoromethyl, trifluoromethyl, C₁₋₄alkyl, C₂₋₄ alkenyl, C₂₋₄alkynyl, C₃₋₆ cycloalkyl, C₃₋₆heterocycloalkyl, NR²³R²⁴ and OR²³;
where R²³ is hydrogen, trifluoromethyl, C₁₋₁₀alkyl, C₂₋₁₀alkenyl, C₂₋₁₀alkynyl, C₃₋₁₀cycloalkyl, C₃₋₁₀cycloalkylalkyl, aryl, arylalkyl, heteroaryl, heteroarylalkyl, heterocyclyl, heterocyclylalkyl, phosphate or an amino acid residue, wherein any of said alkyl, alkenyl, alkynyl, cycloalkyl, aryl, arylalkyl, heteroaryl, heteroarylalkyl, heterocyclyl and heterocyclylalkyl portions are optionally substituted with one or more groups independently selected from oxo (with the proviso that it is not substituted on an aryl or heteroaryl), halogen, cyano, nitro, trifluoromethyl, difluoromethoxy, trifluoromethoxy, azido, —NR²¹SO₂R²⁹, —SO₂NR²¹R²⁸, —C(O)R²¹, C(O)OR²¹, —OC(O)R²¹, —NR²¹C(O)OR²⁹, —NR²¹C(O)R²⁸, —C(O)NR²¹R²⁸, —SR²¹, —S(O)R²⁹, —SO₂R²⁹, —NR²¹R²⁸, —NR²¹C(O)NR²⁸R³⁰, —NR²¹C(NCN)NR²⁸R³⁰, —OR²¹, aryl, heteroaryl, arylalkyl, heteroarylalkyl, heterocyclyl, and heterocyclylalkyl, or R²³ and R²⁴ together with the atom to which they are attached form a 4 to 10 membered carbocyclic, heteroaryl or heterocyclic ring, wherein any of said carbocyclic, heteroaryl or heterocyclic rings are optionally substituted with one or more groups independently selected from halogen, cyano, nitro, trifluoromethyl, difluoromethoxy, trifluoromethoxy, azido, —NR²¹SO2R²⁹, —SO₂NR²¹R²⁸, —C(O)R²¹, —C(O)OR²¹, —OC(O)R²¹, —NR²¹C(O)OR²⁹, —NR²¹C(O)R²⁸, —C(O)NR²¹R²⁸, —SR²¹, —S(O)R²⁹, —SO₂R²⁹, —NR²¹R²⁸, —NR²¹C(O)NR²⁸R³⁰, —NR²¹C(NCN)NR²⁸R³⁰, —OR²¹, aryl, heteroaryl, arylalkyl, heteroarylalkyl, heterocyclyl, and heterocyclylalkyl;
R²⁴ and R²⁵ independently are hydrogen or C₁₋₆ alkyl; or
R²⁴ and R²⁵ together with the atom to which they are attached form a 4 to 10 membered carbocyclic, heteroaryl or heterocyclic ring, wherein said alkyl or any of said carbocyclic, heteroaryl and heterocyclic rings are optionally substituted with one or more groups independently selected from halogen, cyano, nitro, trifluoromethyl, difluoromethoxy, trifluoromethoxy, azido, —NR²¹SO₂R²⁹, —SO₂NR²¹R²⁸, —C(O)R²¹, C(O)OR²¹, —OC(O)R²¹, —NR²¹C(O)OR²⁹, —NR²¹C(O)R²⁸, —C(O)NR²¹R²⁸, —SR²¹, —S(O)R²⁹, —SO₂R²⁹, —NR²¹R²⁸, —NR²¹C(O)NR²⁸R³⁰, —NR²¹C(NCN)NR²⁸R³⁰, —OR²¹, aryl, heteroaryl, arylalkyl, heteroarylalkyl, heterocyclyl, and heterocyclylalkyl;
R²⁶ is trifluoromethyl, C₁₋₁₀alkyl, C₃₋₁₀cycloalkyl, aryl, arylalkyl, heteroaryl, heteroarylalkyl, heterocyclyl or heterocyclylalkyl, wherein any of said alkyl, cycloalkyl, aryl, arylalkyl, heteroaryl, heteroarylalkyl, heterocyclyl and heterocyclylalkyl portions are optionally substituted with one or more groups independently selected from oxo (with the proviso that it is not substituted on an aryl or heteroaryl), halogen, cyano, nitro, trifluoromethyl, difluoromethoxy, trifluoromethoxy, azido, —NR²¹SO₂R²⁹, —SO₂NR²¹R²⁸, —C(O)R²¹, C(O)OR²¹, —OC(O)R²¹, —NR²¹C(O)OR²⁹, —NR²¹C(O)R²⁸, —C(O)NR²¹R²⁸, —SR²¹, —S(O)R²⁹, —SO₂R²⁹, —NR²¹R²⁸, —NR²¹C(O)NR²⁸R³⁰, —NR²¹C(NCN)NR²⁸R³⁰, —OR²¹, aryl, heteroaryl, arylalkyl, heteroarylalkyl, heterocyclyl, and heterocyclylalkyl;
R²¹, R²⁸ and R³⁰ independently are hydrogen, lower alkyl, lower alkenyl, aryl and arylalkyl, and R²⁹ is lower alkyl, lower alkenyl, aryl and arylalkyl;
or any two of R²¹, R²⁸, R³⁰ or R²⁹ together with the atom to which they are attached form a 4 to 10 membered carbocyclic, heteroaryl or heterocyclic ring, wherein any of said alkyl, alkenyl, aryl, arylalkyl carbocyclic rings, heteroaryl rings or heterocyclic rings are optionally substituted with one or more groups independently selected from halogen, cyano, nitro, trifluoromethyl, difluoromethoxy, trifluoromethoxy, azido, aryl, heteroaryl, arylalkyl, heteroarylalkyl, heterocyclyl, and heterocyclylalkyl;
m is 0, 1, 2, 3, 4 or 5; and j is 0, 1 or 2;
X is OR⁶, SR⁶, —NR⁶R⁵, —N(R¹²)OR⁶, —N(R⁵)SO₂R⁶, C₃₋₁₀cycloalkyl, C₁₋₁₀alkyl, aryl, heteroaryl or heterocyclyl,
wherein R⁶ is a group as defined above for R²³;
R⁵ and R¹² are groups as defined above for a group R²⁴; or
R¹² is linked to R⁶ so as to form a protected derivative thereof;
which method comprises hydrolysis of a compound of formula (II)

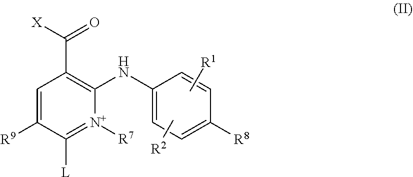

where X, R¹, R², R⁷, R⁸ and R⁹ are as defined above, and L is a leaving group.

Suitable leaving groups L include halogen (such as chlorine, bromine or iodine), as well as many oxygen-linked leaving groups, for example groups of formula OR¹⁰ OC(O)R¹¹ or OSO₂R¹¹ where R¹⁰ is an alkyl group, and R¹¹ is an alkyl or aryl group.

According to a further aspect of the invention, there is provided a method for preparing a compound of formula (IB)

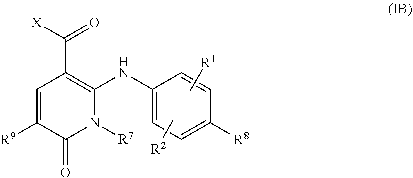

or a pharmaceutically acceptable salt thereof;
where:
R⁷ is methyl or ethyl, either of which are optionally substituted with one or more fluorine atoms;
R¹, R², R⁸ and R⁹ are independently hydrogen, hydroxy, halogen, cyano, nitro, trifluoromethyl, difluoromethyl, fluoromethyl, fluoromethoxy, difluoromethoxy, trifluoromethoxy, azido, —SR²¹, —OR²³, —C(O)R²³, —C(O)OR²³, —NR²⁴C(O)OR²⁶, —OC(O)R²³, —NR²⁴SO₂R²⁶, —SO₂NR²³R²⁴, —NR²⁴C(O)R²³, —C(O)NR²³R²⁴, —NR²⁵C(O)NR²³R²⁴, —NR²⁵C(NCN)NR²³R²⁴, —NR²³R²⁴, C₁₋₁₀ alkyl, C₂₋₁₀ alkenyl, C₂₋₁₀alkynyl, C₃₋₁₀cycloalkyl, C₃₋₁₀cycloalkylalkyl, —S(O)ⱼC₁₋₆alkyl, —S(O)ⱼ(CR²⁴R²⁵)ₘ-aryl, aryl, arylalkyl, heteroaryl, heteroarylalkyl, heterocyclyl, heterocyclylalkyl, —O(CR²⁴R²⁵)ₘaryl, —NR²⁴(CR²⁴R²⁵)ₘ-aryl, —O(CR²⁴R²⁵)ₘ-heteroaryl, —NR²⁴(CR²⁴R²⁵)ₘ-heteroaryl, —O(CR²⁴R²⁵)ₘ-heterocyclyl or —NR²⁴(CR²⁴R²⁵)ₘ-heterocyclyl, wherein any of said alkyl, alkenyl, alkynyl, cycloalkyl, aryl, arylalkyl, heteroaryl, heteroarylalkyl, heterocyclyl and heterocyclylalkyl portions are optionally substituted with one or more groups independently selected from oxo (with the proviso that it is not substituted on an aryl or heteroaryl), halogen, cyano, nitro, trifluoromethyl, difluoromethoxy, trifluoromethoxy, azido, —NR²⁴SO₂R²⁶, —SO₂NR²³R²⁴, —C(O)R²³, —C(O)OR²³, —OC(O)R²³, —NR²⁴C(O)OR²⁶, —NR²⁴C(O)R²³, —C(O)NR²³R²⁴, —NR²³R²⁴, —NR²⁵C(O)NR²³R²⁴, —NR²⁵C(NCN)NR²³R²⁴, —OR²³, aryl, heteroaryl, arylalkyl, heteroarylalkyl, heterocyclyl, and heterocyclylalkyl, and wherein said aryl, heteroaryl, arylalkyl, heteroarylalkyl, heterocyclyl or heterocyclylalkyl rings may be further substituted with one or more groups selected from halogen, hydroxyl, cyano, nitro, azido, fluoromethyl, difluoromethyl, trifluoromethyl, C₁₋₄alkyl, C₂₋₄ alkenyl, C₂₋₄alkynyl, C₃₋₆ cycloalkyl, C₃₋₆heterocycloalkyl, NR²³R²⁴ and OR²³;
where R²³ is hydrogen, trifluoromethyl, C₁₋₁₀alkyl, C₂₋₁₀alkenyl, C₂₋₁₀alkynyl, C₃₋₁₀cycloalkyl, C₃₋₁₀cycloalkylalkyl, aryl, arylalkyl, heteroaryl, heteroarylalkyl, heterocyclyl, heterocyclylalkyl, phosphate or an amino acid residue, wherein any of said alkyl, alkenyl, alkynyl, cycloalkyl, aryl, arylalkyl, heteroaryl, heteroarylalkyl, heterocyclyl and heterocyclylalkyl portions are optionally substituted with one or more groups independently selected from oxo (with the proviso that it is not substituted on an aryl or heteroaryl), halogen, cyano, nitro, trifluoromethyl, difluoromethoxy, trifluoromethoxy, azido, —NR²¹SO2R²⁹, —SO₂NR²¹R²⁸, —C(O)R²¹, C(O)OR²¹, —OC(O)R²¹, —NR²¹C(O)OR²⁹, —NR²¹C(O)R²⁸, —C(O)NR²¹R²⁸, —SR²¹, —S(O)R²⁹, —SO₂R²⁹, —NR²¹R²⁸, —NR²¹C(O)NR²⁸R³⁰, —NR²¹C(NCN)NR²⁸R³⁰, —OR²¹, aryl, heteroaryl, arylalkyl, heteroarylalkyl, heterocyclyl, and heterocyclylalkyl, or R²³ and R²⁴ together with the atom to which they are attached form a 4 to 10 membered carbocyclic, heteroaryl or heterocyclic ring, wherein any of said carbocyclic, heteroaryl or heterocyclic rings are optionally substituted with one or more groups independently selected from halogen, cyano, nitro, trifluoromethyl, difluoromethoxy, trifluoromethoxy, azido, —NR²¹SO2R²⁹, —SO₂NR²¹R²⁸, —C(O)R²¹, —C(O)OR²¹, —OC(O)R²¹, —NR²¹C(O)OR²⁹, —NR²¹C(O)R²⁸, —C(O)NR²¹R²⁸, —SR²¹, —S(O)R²⁹, —SO₂R²⁹, —NR²¹R²⁸, —NR²¹C(O)NR²⁸R³⁰, —NR²¹C(NCN)NR²⁸R³⁰, —OR²¹, aryl, heteroaryl, arylalkyl, heteroarylalkyl, heterocyclyl, and heterocyclylalkyl;
R²⁴ and R²⁵ independently are hydrogen or C₁₋₆ alkyl; or
R²⁴ and R²⁵ together with the atom to which they are attached form a 4 to 10 membered carbocyclic, heteroaryl or heterocyclic ring, wherein said alkyl or any of said carbocyclic, heteroaryl and heterocyclic rings are optionally substituted with one or more groups independently selected from halogen, cyano, nitro, trifluoromethyl, difluoromethoxy, trifluoromethoxy, azido, —NR²¹SO₂R²⁹, —SO₂NR²¹R²⁸, —C(O)R²¹, C(O)OR²¹, —OC(O)R²¹, —NR²¹C(O)OR²⁹, —NR²¹C(O)R²⁸, —C(O)NR²¹R²⁸, —SR²¹, —S(O)R²⁹, —SO₂R²⁹, —NR²¹R²⁸, —NR²¹C(O)NR²⁸R³⁰, —NR²¹C(NCN)NR²⁸R³⁰, —OR²¹, aryl, heteroaryl, arylalkyl, heteroarylalkyl, heterocyclyl, and heterocyclylalkyl;
R²⁶ is trifluoromethyl, C₁₋₁₀alkyl, C₃₋₁₀cycloalkyl, aryl, arylalkyl, heteroaryl, heteroarylalkyl, heterocyclyl or heterocyclylalkyl, wherein any of said alkyl, cycloalkyl, aryl, arylalkyl, heteroaryl, heteroarylalkyl, heterocyclyl and heterocyclylalkyl portions are optionally substituted with one or more groups independently selected from oxo (with the proviso that it is not substituted on an aryl or heteroaryl), halogen, cyano, nitro, trifluoromethyl, difluoromethoxy, trifluoromethoxy, azido, —NR²¹SO₂R²⁹, —SO₂NR²¹R²⁸, —C(O)R²¹, C(O)OR²¹, —OC(O)R²¹, —NR²¹C(O)OR²⁹, —NR²¹C(O)R²⁸, —C(O)NR²¹R²⁸, —SR²¹, —S(O)R²⁹, —SO₂R²⁹, —NR²¹R²⁸, —NR²¹C(O)NR²⁸R³⁰, —NR²¹C(NCN)NR²⁸R³⁰, —OR²¹, aryl, heteroaryl, arylalkyl, heteroarylalkyl, heterocyclyl, and heterocyclylalkyl;
R²¹, R²⁸ and R³⁰ independently are hydrogen, lower alkyl, lower alkenyl, aryl and arylalkyl, and R²⁹ is lower alkyl, lower alkenyl, aryl and arylalkyl;
or any two of R²¹, R²⁸, R³⁰ or R²⁹ together with the atom to which they are attached form a 4 to 10 membered carbocyclic, heteroaryl or heterocyclic ring, wherein any of said alkyl, alkenyl, aryl, arylalkyl carbocyclic rings, heteroaryl rings or heterocyclic rings are optionally substituted with one or more groups independently selected from halogen, cyano, nitro, trifluoromethyl, difluoromethoxy, trifluoromethoxy, azido, aryl, heteroaryl, arylalkyl, heteroarylalkyl, heterocyclyl, and heterocyclylalkyl;
m is 0, 1, 2, 3, 4 or 5; and j is 0, 1 or 2;
X is OR⁶, —NR⁶R⁵, —N(R¹²)OR⁶, —N(R⁵)SO₂R⁶, C₃₋₁₀cycloalkyl, C₁₋₁₀alkyl, aryl, heteroaryl or heterocyclyl;
wherein R⁶ is a group as defined above for R²³;
R⁵ and R¹² are groups as defined above for a group R²⁴; or
R¹² is linked to R⁶ so as to form a protected derivative thereof;
which method comprises hydrolysis of a compound of formula (IIB)

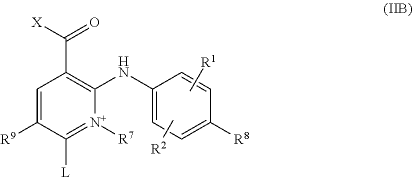

where X, R¹, R², R⁷, R⁸ and R⁹ are as defined above, and L is a leaving group.

The reaction is suitably effected by reaction in aqueous medium, with or without an organic co-solvent, such as an alcoholic solvent, such as ethanol or IMS (industrial methylated spirit), at temperatures of from ambient temperature to the boiling point of the solvent, for example a temperature of from 45 to 65° C. was found to be convenient.

Compounds of formula (II) are suitably prepared by reacting a compound of formula (III)

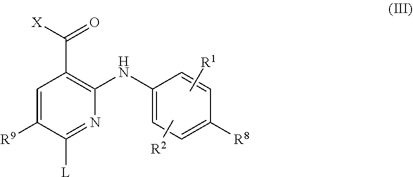

where L, X, R¹, R², R⁸ and R⁹ are as defined in relation to formula (I); with a compound of formula (IV)

R⁷-L¹ (IV)

where R⁷ is as defined in relation to formula (I) and L¹ is a leaving group.

Suitable leaving groups L¹ include in particular O-linked groups such as alkoxy, OCOalkyl, OCOaryl, OSO₂alkyl, OSO₂aryl as well as halogen atoms. Thus examples of groups L¹ are trifluoromethanesulfonate, mesylate or tosylate as well as halogen such as Cl, Br or I.

The reaction is suitably effected in an unreactive organic solvent such as chlorobenzene. It has been found that pyridines of type (III) are not particularly reactive and alkylation reactions can be slow. Thus, it is beneficial to carry out the reaction at an elevated temperature, for example between 60 to 100° C., and at high concentration of the compound of formula (V), for example between 3-10 Kg/L, typically around 4 Kg/L, in order for the reaction to proceed at a convenient rate.

Although not essential, where available, the introduction of a seed charge will facilitate isolation of the compound of formula (II) during this procedure.

Compounds of formula (II) obtained in this way are suitably used directly in the preparation of compounds of formula (I). Extensive drying or purification stages have been found not to be necessary, although a crude separation on a filter is helpful.

Compounds of formula (III) may themselves be prepared by a process comprising reacting a compound of formula (V)

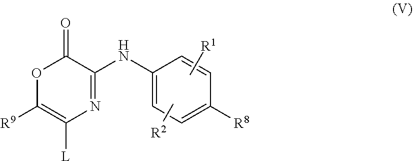

where L, R¹, R², R⁸ and R⁹ are as defined in relation to formula (I);
with a compound of formula (VI) or (VIa)

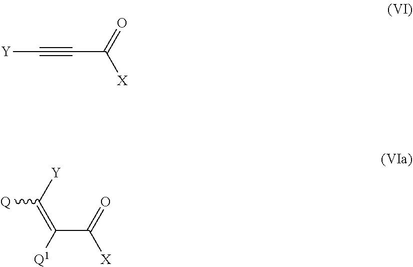

where X is as defined in relation to formula (I), Y is hydrogen or a removable group (such as SiR¹⁹R²⁰R²¹ or SnR¹⁹R²⁰R²¹ where R¹⁹, R²⁰ and R²¹ are independently selected from hydrogen or C₁₋₆alkyl (such as methyl) or aryl and Q and Q¹ are independently selected from hydrogen or a group which is readily removeable by elimination such as OC₁₋₆alkyl, OCOC₁₋₆alkyl, mesylate, tosylate or halogen (e.g. chlorine, bromine or fluorine). For example, one of Q or Q¹, for instance Q¹, is hydrogen and the other is a removeable group as defined above such as halogen.

When a compound of formula (VIa) is used, the initial product of the reaction between the compound of formula (V) and the compound of formula (VIa), is a dihydroyridine, which is subsequently aromatised by the elimination of a removable group and hydrogen, so one of Q or Q¹ must be hydrogen and the other of Q or Q¹ must be a removable group. Examples are shown in the scheme below.

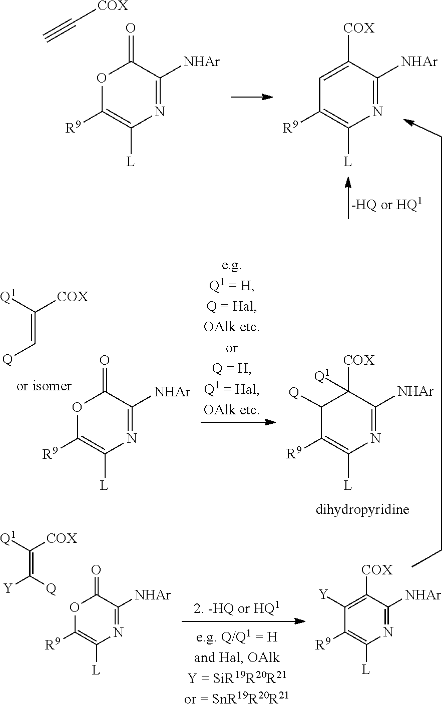

In another embodiment, if a compound of formula (VIa) is used where Y, Q and Q¹ are all hydrogen, the product of the reaction between a compound of formula (V) and a compound of formula (VIa) is a dihydropyridine which is readily oxidised to form a pyridine of formula (III).

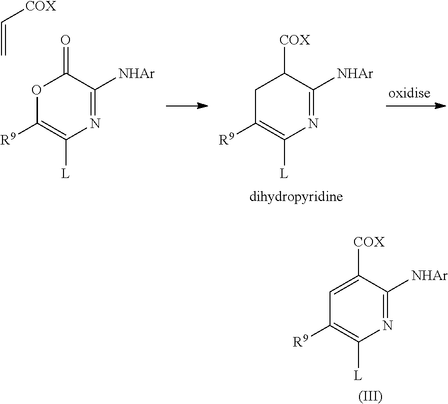

Reactions of this type are commonly known as Diels-Alder cycloaddition reactions and the component (VI) or (VIa) is know as the dienophile. The unsymmetrical dienophiles such as (VI) and (VIa) can react in two regiochemical modes, leading to separate isomeric products and this is highly inconvenient and wasteful for a large-scale manufacturing process. However, the applicants have found that a high level of control of the reaction may be achieved when novel anilides of structure (V) are used in the Diels-Alder process.

Furthermore, intermediates (III) are generally found to be highly crystalline and under appropriate conditions, as described below, they are isolated in very high yield and high purity by simple crystallisation from the reaction mixture. Thus the inclusion of this step is highly advantageous in the formulation of a manufacturing process.

The reaction can be suitably effected by heating the substrates together, with or without solvent, for example between 50-200° C. Suitable solvents including toluene, acetonitrile, anisole, chlorobenzene, isopropanol and n-butyl acetate.

However, as the reaction is a 4+2 cycloaddition process, it is a thermal process that is accelerated by heating. As such, using a solvent with high boiling point allows the reaction to be conveniently carried out at a high temperature, which brings about the rapid completion of the reaction, without the need for a large excess of either substrate. In addition to this we have found that by carefully choosing a solvent in which the product is sparingly soluble, it can be isolated by simply cooling the reaction mixture at the end of reaction and filtering off the product from the solvent. Aromatic solvents, such as toluene are generally useful for these processes and an anti-solvent, such as a saturated hydrocarbon, can be added at the end of the reaction in order to increase the product recovery.

ⁿButyl acetate is particularly effective as a single solvent in some cases, for example when compound (V) (R₁═F, R₂═H, R₈═I, R₉=Me, L=Cl) is reacted with ethyl propiolate, in ⁿbutyl acetate, at about 120° C., for 6 h and the mixture is then cooled to about 0° C., the chloropyridine product is isolated in >90% yield, with an assay of nearly 100% i.e. greater than 99% purity with no detectable by-products above the limit of 0.05% as determined by HPLC.

To further illustrate the generality of this approach for the preparation of substituted pyridines a range of reactions were performed as shown in the Table. The yields are unoptimised.

| | | | Rxn | Yield |
|---|---|---|---|---|
| Diene | Alkyne | Product | Time | (%) |
| 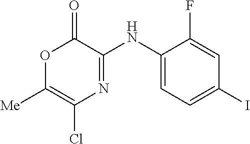 | 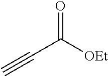 | 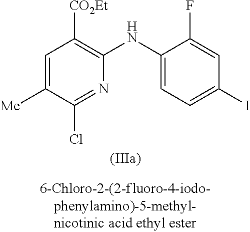 | 6 h | 89% |
| 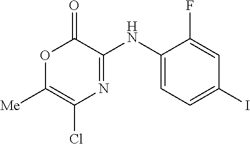 | 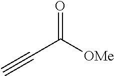 | 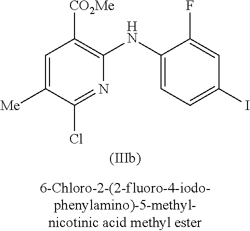 | 19 | 76 |
| 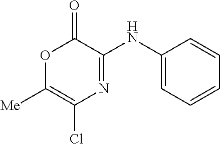 | 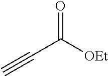 | 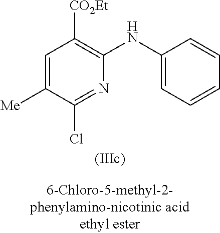 | 21 h | 63 |
| 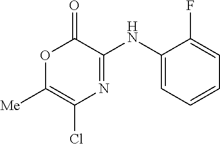 | 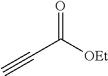 | 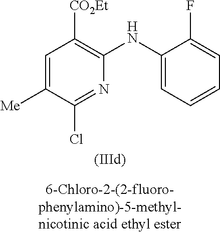 | 21 h | 85 |
| 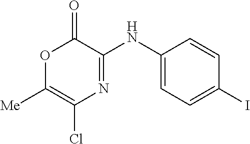 | 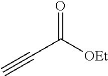 | 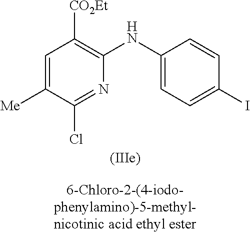 | 21 h | 89 |

Compounds of formula (V) are conveniently prepared by reacting a compound of formula (VII)

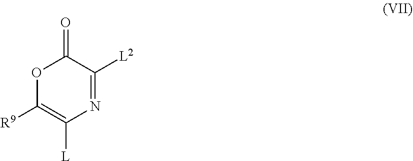

where R⁹ is as defined in relation to formula (I) and L² is a leaving group, with a compound of formula (VIII)

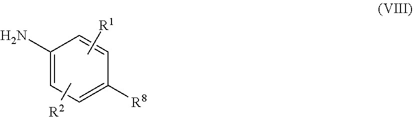

where R¹, R² and R⁸ are as defined in relation to formula (I).

Suitable leaving groups L² include trifluoromethanesulfonate, mesylate or tosylate as well as halogen such as Cl, Br, I.

The reaction of anilines with compound (VII) is surprisingly selective, even when L and L² are the same leaving group. A wide variety of aromatic amines are suitable for this process. In particularly reactions with substituted anilines are effective, more particularly the process is suited to anilines such as, aniline; 2-fluoroaniline; 2-fluoro-4-iodoaniline and 4-iodoaniline.

The reaction is suitably effected in an organic solvent such as tetrahydrofuran, toluene, dioxane, isopropanol, suitably at temperatures from ambient to 100° C., more conveniently at 75-85° C., with or without the mediation of an acid or Lewis acid catalyst. In ethereal solvents, such as tetrahydrofuran the use of a Lewis acid catalyst, such as boron trifluoride is particularly effective and in an aromatic solvent, such as toluene or chlorobenzene, acid catalysts, such as methanesulfonic acid are beneficial. In one embodiment of the invention R⁹ is methyl and L and L² are Cl. In another aspect of the invention R⁹ is methyl, L and L² are Cl, R¹ is F, R² is H and R⁸ is I.

In one aspect, the intermediate (VII) is prepared in situ from a compound of formula (IX) and a compound of formula (X)

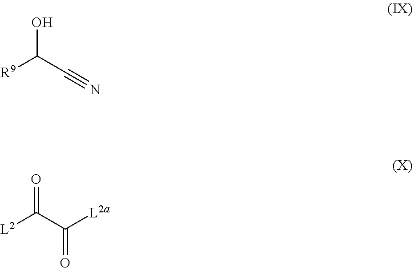

where R⁹ and L² are as defined above, and L^{2a} is a leaving group, as defined above for L² or is OH. In one aspect, R⁹ in compound (IX) is methyl and compound (X) is oxalyl chloride.

Suitably the above compounds are reacted together in a solvent that is compatible with the following step of the process, with or without an amine hydrochloride catalyst. Aromatic solvents such as toluene, xylene and chlorobenzene are suitable and toluene is particularly effective. Although the reaction can be carried out at a range of temperatures, maintaining the temperature between 60-80° C., is convenient and effective. Before the subsequent stage of the reaction is carried out, it is useful to remove any remaining compound (X) by quenching the mixture with water and a suitably chosen reaction solvent, such as toluene, which may then allow the removal of water by azeotropic distillation. The resultant solution contains compound (VII) and this is reacted directly with a compound (VIII) as described above, preferably with an acid catalysts, such as methanesulfonic acid. In one aspect, the aniline of formula VIII is 2-fluoro-4-iodoaniline.

To further illustrate the generality of this approach for the preparation of 2-phenylamino substituted 5-Chloro-6-methyl-[1,4]oxazin-2-ones a range of reactions were performed as shown in the Table. The yields are unoptimised.

| | | | Rxn | Yield |
|---|---|---|---|---|
| [1,4]-Oxazin-2-one | Aniline | Product | Time | (%) |
| 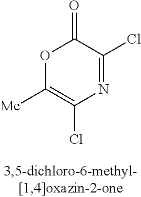 | 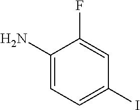 | 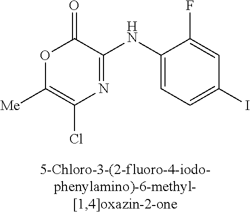 | 39 h | 80% |
| 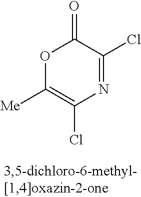 | 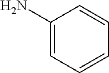 | 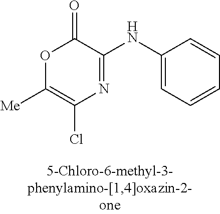 | 48 h | 36* |
| 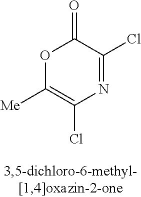 | 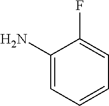 | 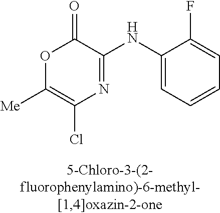 | 45 h | 64 |
| 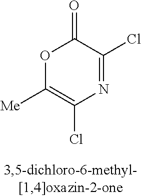 | 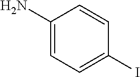 | 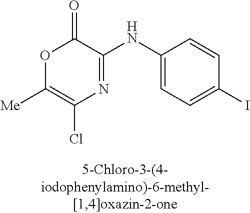 | 45 h | 67 |
| 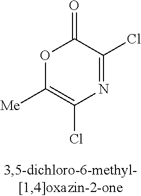 | 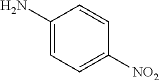 | 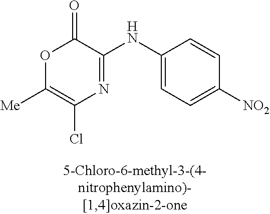 | 48 h | 89% |

*Yield compromised by spillage of material.

In one aspect, the invention provides a process for the formation of pyridones of formula (I)) comprising the steps 1) to 4):

1) formation of a compound of formula (V)

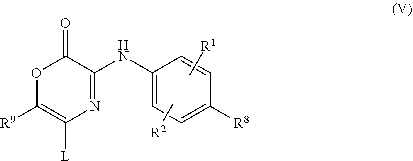

where L, R¹, R², R⁸ and R⁹ are as defined in relation to formula (I); by coupling of compounds of formulae (VII) and (VIII) and suitably incorporating a process where a compound of formula (VII) can be prepared in situ from compounds of formulae (IX) and (X);

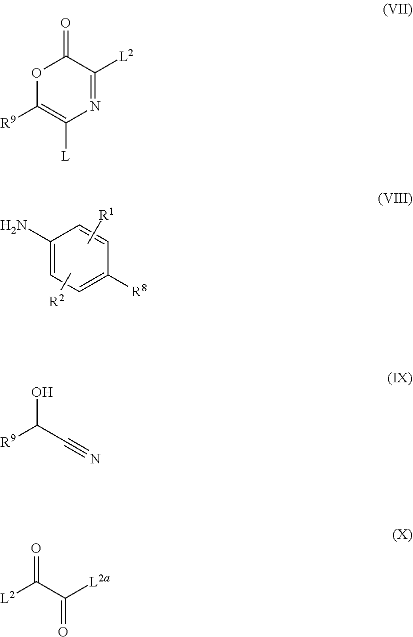

2) 4+2 cycloaddition reaction of a compound of formula (V) with propionic acid or a derivative thereof to form a pyridine of formula (III);

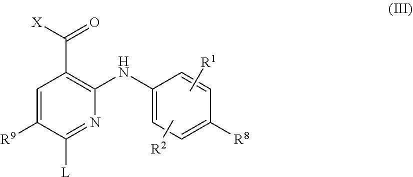

3) alkylation of a pyridine of formula (III) to form a pyridinium salt of formula (II); and

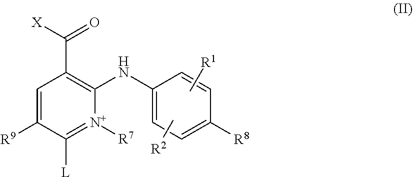

4) hydrolysis of a compound of formula (II) to provide a compound of formula (I)

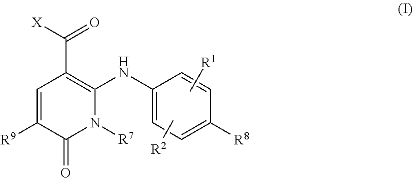

This combination of steps represents a highly selective route for the preparation of pyridones of formula (I), which circumvent the selectivity and chemical hazards issues highlighted above.

Compounds of formula (VII) and (VIII) are known compounds and may be prepared by conventional methods. For example preparation of 3,5-dichloro-6-methyl-[1,4]oxazin-2-one (VIIa) and related compounds was reported by Hoornaert et al. Tetrahedron, 1994, 5211; Synthesis 1991, 765; Tetrahedron Lett., 1989, 3183. All the anilines used, for example (VIIa), were obtained from commercial suppliers.

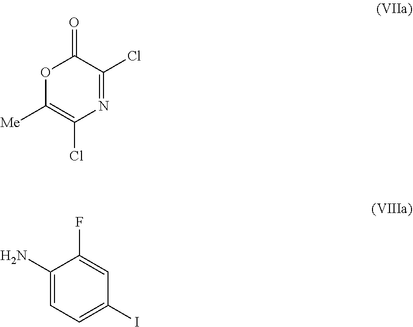

Compounds (IX) and (X) are commercially available in bulk, or can be produced from known compounds by conventional methods, and are, for example, oxallyl chloride and lactonitrile, both obtained from commercial suppliers.

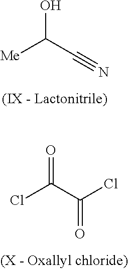

Compounds of formulae (II), (III) and (V) as defined above are novel and form a further aspect of the invention.

In one embodiment, R¹ and R² are independently selected from hydrogen, halogen, cyano, nitro, trifluoromethyl, difluoromethoxy, trifluoromethoxy, azido, amino, C₁₋₆-alkylamino, di-C₁₋₆alkylamino, C₁₋₁₀alkyl, C₂₋₁₀alkenyl, C₂₋₁₀alkynyl, C₁₋₁₀alkoxy, C₁₋₁₀alkylcarbonyl, carbamoyl, C₁₋₆alkylcarbamoyl, di-C₁₋₆alkylcarbamoyl, sulphamoyl, C-₁₋₆alkylsulphamoyl, di-C₁₋₆alkylsulphamoyl, C₁₋₁₀thioalkyl.

In one aspect, R¹ and R² are independently selected from hydrogen, halogen, C₁₋₆alkyl, OCH₃ or SCH₃.

In one aspect, in the compounds defined above, R² is hydrogen.

Suitably R¹ is other than hydrogen, and is in one aspect, halogen.

In addition, in one aspect, R¹ is a substituent ortho to the amine group and meta to the R⁸ group.

Thus, particular compounds of formula (I) are compounds of formula (IA)

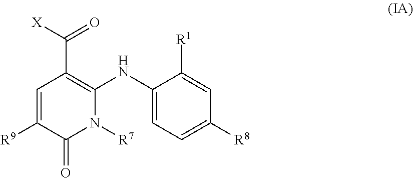

where X, R¹, R⁸, R⁷ and R⁹ are as defined in relation to formula (I).
Similarly particular compounds of formula (II), (III) and (V) are compounds of formula (IIA), (IIIA) and (VA) respectively.

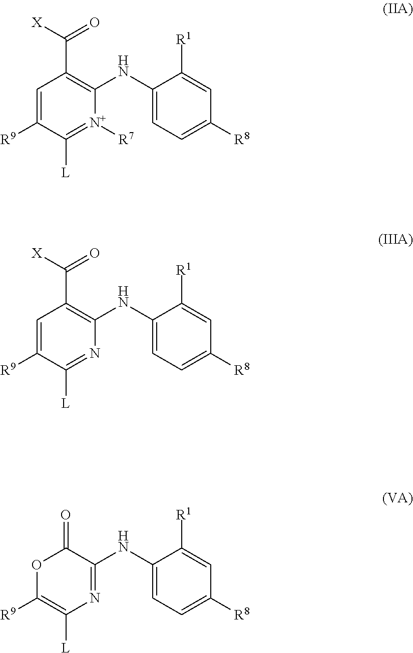

where X, R¹, R⁸, R⁷ and R⁹ are as defined in relation to formula (I).

Suitably, R⁸ in the above compounds is hydrogen, halogen (e.g. F, Cl, Br, I), C₁₋₆alkyl, C₁₋₆alkoxy or C₁₋₆thioalkyl. For instance, R⁸ is hydrogen, fluorine, chlorine, bromine, iodine C₁₋₄alkyl, OCH₃ or SCH₃.

Particular examples of R⁸ in compounds of formula (I), (II), (III) and (V), as well as (IA), (IIA), (IIIA) and (VA) is iodine.

Particular examples of R¹ in compounds of formula (I), (II), (III) and (V) as well as (IA) (IIA) (IIIA) and (VA) is fluorine.

Particular examples of R⁷ in compounds of formula (I) and (II) as well as (IA) and (IIA) are methyl, trifluoromethyl or ethyl, in particular methyl.

In one aspect, R⁹ is hydrogen, CN, halogen, (e.g. F, Cl, Br, I), or C₁₋₄alkyl optionally substituted by one or more groups independently selected from F or CN.

In one aspect R⁹ in compounds of formula (I), (II), (III) and (V) as well as (IA), (IIA), (IIIA) and (VA) are C₁₋₄alkyl optionally substituted by one or more groups independently selected from F or CN. In one aspect, R⁹ is unsubstituted C₁₋₄alkyl such as methyl or ethyl, and especially methyl.

In a particular embodiment, X is OR⁶, NHR⁶, —N(R¹²)OR⁶, SR⁶ or CH₂R⁶, where R⁶ is as defined above. In one aspect, R⁶ is selected from hydrogen, or C₁₋₁₀alkyl optionally substituted by hydroxy or cycloalkyl. R¹² is suitably also selected from hydrogen, or C₁₋₁₀alkyl optionally substituted by hydroxy or cycloalkyl.

Suitably when R¹² is linked to R⁶ to form a protected derivative of R⁶, it is in the form of an aza-acetal derivative, for example of sub-formula (i)

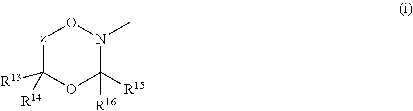

where z is a group (CR¹⁷R¹⁸)_{q} where q is 0, 1 or 2, R¹³, R¹⁴, R¹⁵, R¹⁶ and each R¹⁷ and R¹⁸ are independently selected from hydrogen or C₁₋₄alkyl, in particular hydrogen or methyl. Acid hydrolysis, for example using aqueous mineral acid such as hydrochloric acid, of the compound will result in ring opening of the group of formula (I) to form a group of (ii)

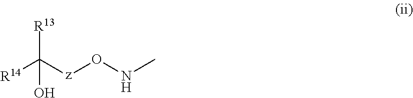

with expulsion of an ketone such as acetone.

In the compounds of formula (I), (II), (III), (IA), (IIA) and (IIIA), suitably X is OR⁶, —NHR⁶, or —NOR⁶.

In one aspect, X is OR⁶.

Particular examples of R⁶ in compounds of formula (I), (II) and (III) as well as (IA), (IIA), and (IIIA) are methyl or ethyl, in particular methyl.

However, in an alternative embodiment, the group X is a group —NHR⁶ where R⁶ is R^{b} and R^{b} is as defined above in relation to formula (A).

It is to be understood that, insofar as certain of the compounds of formula I defined above may exist in optically active or racemic forms by virtue of one or more asymmetric carbon atoms, the invention includes in its definition any such optically active or racemic form which possesses the above-mentioned activity. The synthesis of optically active forms may be carried out by standard techniques of organic chemistry well known in the art, for example by synthesis from optically active starting materials or by the resolution of a racemic form. Similarly, the above-mentioned activity may be evaluated using the standard laboratory techniques referred to hereinafter.

It is to be understood that certain compounds of formula (I) defined above may exhibit the phenomenon of tautomerism. In particular, tautomerism may affect any heterocyclic groups that bear 1 or 2 oxo substituents. It is also to be understood that the present invention includes in its definition any such tautomeric form, or a mixture thereof, which possesses the above-mentioned activity and is not to be limited merely to any one tautomeric form utilised within the formulae drawings or named in the Examples.

It is to be understood that certain compounds of formula (I) above may exist in unsolvated forms as well as solvated forms, such as, for example, hydrated forms. It is also to be understood that the present invention encompasses all such solvated forms.

It is also to be understood that certain compounds of the formula (I) may exhibit polymorphism. It is also to be understood that the present invention encompasses all such polymorphic forms.

In this specification the generic term “alkyl” includes both straight chain and branched-chain alkyl groups such as propyl, isopropyl and tert-butyl. Unless otherwise stated, they suitably have from 1-10 carbon atoms, in particular from 1-6 carbon atoms. However references to individual alkyl groups such as “propyl” are specific for the straight-chain version only, references to individual branched-chain alkyl groups such as “isopropyl” are specific for the branched-chain version only. An analogous convention applies to other generic terms, for example (1-4C)alkoxy includes methoxy, ethoxy and isopropoxy.

The term “halo” or “halogen” refers to fluoro, chloro, bromo, or iodo.

The term “aryl” refers to carbocyclic aromatic groups, such as phenyl or naphthyl, but in particular phenyl.

The term “heterocyclic” or “heterocyclyl”, unless otherwise defined herein, refers to saturated, partially saturated or unsaturated monocyclic rings containing 4, 5, 6 or 7 ring atoms wherein at least one of said atoms, and suitably from 1-4 or said atoms, is a heteroatom, such as oxygen, sulphur or nitrogen. Where they are unsaturated, they may be aromatic, and such rings are described as “heteroaryl” groups.

In particular compounds of the invention, “heterocyclic rings” are saturated monocyclic rings that contain 4, 5, 6 or 7 ring atoms, and especially 5 or 6 ring atoms.

Examples and suitable values of the term “heterocyclic ring” used herein are pyrrolidinyl, imidazolidinyl, pyrazolidinyl, piperidinyl, piperazinyl, morpholin-4-yl, thiomorpholin-4-yl, 1,4-oxazepan-4-yl, diazepanyl and oxazolidinyl.

Examples of “heteroaryl” rings include thienyl, furanyl, pyrrolyl, imidazolyl, pyrazolyl, thiazolyl, isothiazolyl, isoxazolyl, oxazolyl, thiadiazolyl, oxadiazolyl, triazolyl, tetrazolyl, pyridinyl, pyrazinyl, pyridazinyl or pyrimidinyl.

An important feature of this route to compounds of formula (I) is that, in general, no protecting groups are required when the processes are carried out as described. The type of steps involved mean that this is a viable option in most instances, which is an advantage in terms of reducing the complexity of the reaction and improving the efficiency.

However, it will be appreciated by a person skilled in the art that in some of the reactions mentioned herein it may be desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Furthermore, the synthesis of optically active forms may be carried out by standard techniques of organic chemistry well known in the art, for example by synthesis from optically active starting materials or by resolution of a racemic form.

The invention will now be illustrated in the following Examples in which, generally:

(i) temperatures are given in degrees Celsius (° C.); operations were carried out at room or ambient temperature, that is, at a temperature in the range of 18 to 25° C.;
(ii) evaporation of solvent was carried out using a rotary evaporator under reduced pressure (600 to 4000 Pascals; 4.5 to 30 mmHg) with a bath temperature of up to 60° C.;
(iii) in general, the course of reactions was followed by HPLC and/or analytical LC-MS, and reaction times are given for illustration only. The retention times (t_{R}) were measured on an Agilent 1100 HPLC instrument or an Agilent 1100 MSD single quadrupole LC-MS with electrospray ionisation, with a 50×4.6 mm Zorbax SB-C18 1.8 μm column: detection UV 250 nM and MS; flow rate 1.25 mL min⁻1; linear gradient from 65% water:25% methanol containing 10% TFA to 25% water:65% methanol containing 10% TFA over 13.5 minutes; column temp. 40° C. Accurate mass measurements were carried out on a AC113, Bruker MicroTOFQ with AC58-Agilent 1100 LC using APCI detection for accurate determination of mass ions.
(iv) final products had proton nuclear magnetic resonance (NMR) spectra and mass spectral data;
(v) yields are given for illustration only and are not necessarily those which can be obtained by diligent process development; preparations were repeated if more material was required;
(vi) when given, NMR data is in the form of delta values for major diagnostic protons, given in parts per million (ppm) relative to tetramethylsilane (TMS) as an internal standard, determined at 400 MHz using perdeuterio dimethyl sulfoxide (DMSO-d₆) as solvent unless otherwise indicated; the following abbreviations have been used: s, singlet; bs, broad singlet; d, doublet; dd, doublet of doublets; t, triplet; at, apparent triplet; q, quartet; m, multiplet; br, broad;
(vii) chemical symbols have their usual meanings; SI units and symbols are used;
(viii) solvent ratios are given in volume:volume (v/v) terms; and
(ix) mass spectra were run by electrospray (ESP) or by atmospheric pressure chemical ionisation (APCI); values for m/z are given; generally, only ions which indicate the parent mass are reported; and unless otherwise stated, the mass ion quoted is (MH)⁺ which refers to the protonated mass ion; reference to M⁺ is either to the mass ion generated by loss of an electron or to the mass ion of a quaternary salt cation; MNa⁺ refers to the Mass ion+Na⁺ and reference to M-H′ is to the mass ion generated by loss of a proton. In addition, the following abbreviations have been used, where necessary:—
LiHMDS Lithium bis(trimethylsilyl)amide
DMSO dimethylsulphoxide
NMP 1-methyl-2-pyrrolidinone
THF tetrahydrofuran;

### DMF N,N-dimethylformamide;

DIPEA di-isopropylethylamine;
IPA isopropyl alcohol;
MTBE Methyl tert-butyl ether

### EXAMPLE 1

### 2-(2-Fluoro-4-iodo-phenylamino)-1,5-dimethyl-6-oxo-1,6-dihydro-pyridine-3-carboxylic acid (Ia)

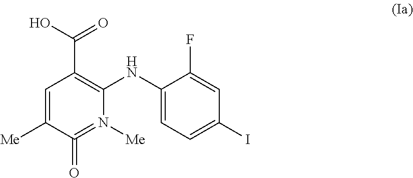

### 2-Chloro-6-(2-fluoro-4-iodo-phenylamino)-5-ethoxycarbonyl-1,3-dimethyl-pyridinium trifluoromethanesulfonate

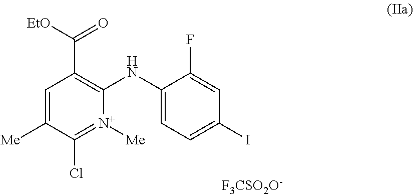

To an agitated slurry of 6-chloro-2-(2-fluoro-4-iodo-phenylamino)-5-methyl-nicotinic acid ethyl ester (IIIa) (156.5 g; 357.5 mmoles) in chlorobenzene, (544 mL), at ambient temperature, was added methyl trifluoromethanesulfonate (103 mL; 150 g; 894 mmoles; 2.5 equiv.), followed by chlorobenzene (78 mL) as a line wash. The mixture was then heated to 90° C., with dissolution of the solids and ˜55° C. After 20 hours at 90° C. the solution was pale orange in colour and HPLC analysis indicated that there was no remaining staring material. The mixture was then cooled to 55° C., over about 1 hour, followed by the addition of water (207 mL; 11.5 moles) over ˜20 min, keeping the temperature of the contents below 62° C. After 10 min a second charge of water (104 mL; 5.78 moles) was added over ˜10 min. During the initial water charge there was an exotherm from 54 to 61° C. and the temperature at the end of the water addition was 51° C. The temperature was then allowed to warm to 55° C. (jacket temperature) and the mixture was biphasic. The agitation rate was reduced and a seed charge of 2-chloro-6-(2-fluoro-4-iodo-phenylamino)-5-ethoxycarbonyl-1,3-dimethyl-pyridinium trifluoromethanesulfonate (IIa) (1.10 g; 1.76 mmoles) was added. (The seed material was made by the route described herein in Example 1 except no seed was added to initiate crystallisation.) After 1 h the mixture was cooled from 55 to 10° C. over 4 hours, then kept at 10° C. for 2 hours, during which time the pyridinium salt crystallized. The resultant slurry was then filtered under vacuum, and the filter cake was washed with isopropyl alcohol (211 mL), then dried by pulling air through for 20 min, to provide 2-chloro-6-(2-fluoro-4-iodo-phenylamino)-5-ethoxycarbonyl-1,3-dimethyl-pyridinium trifluoromethanesulfonate, 180 g (as a yellow IPA damp solid, with an assay of 91.8% w/w, equivalent to 165.2 g @100% w/w, 77% yield), δ_{H} (400 MHz, CDCl₃) 1.39 (3H, t, J7, CH₂CH₃), 2.53 (3H, br. s, ArCH₃), 3.90 (3H, s, NCH₃), 4.35 (2H, q, J7, CH₂CH₃), 7.28 (1H, ˜t, J8.5, ArH), 7.52 (1H, m, ArH), 7.58 (1H, m, ArH), 8.57 (1H, br. s, ArH), 10.69 (s, 1H, NH); δ¹⁹F (CDCl₃) −78.78 (3F, s) −121.85 (1F, d, J9); m/z. (LCMS, ES⁺) 449.0, 451.0 (3:1 M⁺ (Cl=35):M⁺ (Cl=37)).

A stirred slurry of the intermediate (˜180 g) in IMS (industrial methylated spirit) (1.65 L), was heated to 50° C. at which temperature a solution was formed. Sodium hydroxide (447 mL; 465 g; 894 mmoles) was added to the mixture over ˜40 min, then after 10 min a 2nd charge of sodium hydroxide (224 mL; 232 g; 447 mmoles) was added, over ˜20 minutes, to produce a bright orange solution. The mixture was then held at 50° C. for 6 h after which time HPLC analysis indicated that the reaction was complete. Acetonitrile (311 mL) was added to the mixture over ˜15 min, keeping the internal temperature >45° C. and then allowing the temperature to settle at ˜50° C. Dilute hydrochloric acid (84 mL; 840 mmoles) was added over 2 h, and the mixture was then held at 50° C. for 1 h, then filtered under vacuum. The filter cake was washed with a mixture of IMS (222 mL) and water (89 mL), then dried in a vacuum oven at 50° C. overnight, to provide 2-(2-fluoro-4-iodo-phenylamino)-1,5-dimethyl-6-oxo-1,6-dihydro-pyridine-3-carboxylic acid (Ia) (99.9 g, 69% yield over two steps), δ_{H} (400 MHz, D6-DMSO) 2.02 (3H, d, J1, ArCH₃), 3.18 (3H, s, NCH₃), 6.65 (1H, t, J8.5, ArH), 7.44 (1H, bd, J8.5, ArH), 7.69 (1H, dd, J10.5, 2, ArH), 7.77 (1H, m, J1, ArH), 9.59 (1H, br.s, NH), 13.00 (1H, br. s, COOH), m/z (LCMS, ES⁺) 403.0, 425.0 (1:1 MH⁺:MNa⁺).

### EXAMPLE 2

### 2-Chloro-6-(2-fluoro-4-iodo-phenylamino)-5-methoxycarbonyl-1,3-dimethyl-pyridinium trifluoromethanesulfonate

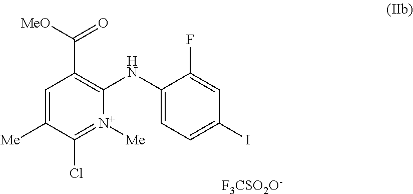

To an agitated slurry of 6-chloro-2-(2-fluoro-4-iodo-phenylamino)-5-methyl-nicotinic acid methyl ester (IIIb) (1.0 g; 2.38 mmoles) in toluene, (5 mL), at ambient temperature, was added methyl trifluoromethanesulfonate (1.3 mL; 2.0 g; 11.9 mmoles; 5.0 equiv.). The mixture was then heated to 85° C. After 22 hours at 85° C. the solution was heated to 90° C. for a further 2 hours, after which the mixture had formed a bi-phase. The lower phase was evaporated to dryness, toluene (5 mL) added and was re-evaporated to dryness. The resulting solid/oil mixture was triturated with MTBE (3 mL) and the resulting solid filtered, washed with MTBE (10 mL) and dried to provide 2-Chloro-6-(2-fluoro-4-iodo-phenylamino)-5-methoxycarbonyl-1,3-dimethyl-pyridinium trifluoromethanesulfonate (760 mg @ 88% w/w, corrected yield 48%), δ_{H} (400 MHz, CDCl₃) 2.53 (3H, s, ArCH₃), 3.91-3.90 (6H, m, NCH₃/COOCH₃), 7.30 (1H, d, J8.5, ArH), 7.53 (1H, dd, J10, 2, ArH), 7.59 (1H, d, J8.5, ArH), 8.58 (1H, s, ArH), 10.63 (1H, s, NH); m/z (LCMS, ES⁺) 435.0, 437.0 (3:1 M⁺ (Cl=35):M⁺ (Cl=37)).

### EXAMPLE 3

### 6-Chloro-2-(2-fluoro-4-iodo-phenylamino)-5-methyl-nicotinic acid ethyl ester (IIIa)

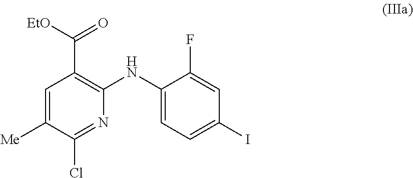

To a stirred slurry of 5-Chloro-3-(2-fluoro-4-iodo-phenylamino)-6-methyl-[1,4]oxazin-2-one (50 g; 131.4 mmoles) in butyl acetate (288 mL) was added ethyl propriolate (15.6 g; 157.7 mmoles; 1.20 equiv), followed by a butyl acetate line wash (12.5 mL). The mixture was then heated to 120° C., with a solution being formed at ˜85° C. After 6 h at 120° C., HPLC analysis indicated that the reaction was complete and the mixture was cooled to 75° C. over 1 h, before seeding with 6-chloro-2-(2-fluoro-4-iodo-phenylamino)-5-methyl-nicotinic acid ethyl ester (461 mg; 1.05 mmoles). (The seed material was made by the route described herein in Example 3 except no seed was added to initiate crystallisation.) The mixture was then held at 75° C. for 1 h then cooled to 0° C. over 5 h and held at that temperature for 2 h. The product was filtered under vacuum and the filter cake was washed with butyl acetate (2×100 mL), and dried in a vacuum oven at 45° C., overnight, to provide 6-chloro-2-(2-fluoro-4-iodo-phenylamino)-5-methyl-nicotinic acid ethyl ester (51.3 g, 89% yield), δ_{H} (400 MHz, CDCl₃) 1.42 (3H, t, J7, CH₂CH₃) 2.30 (3H, s, ArCH₃) 4.41 (2H, q, J7, CH₂CH₃) 7.44 (2H, m, ArH), 8.08 (1H, s, ArH), 8.39 (1H, ˜t, J8.5, ArH), 10.38 (1H, m, NH); m/z. (LCMS, ES⁺) 434.9, 436.9 (3:1 MH⁺ (Cl=35):MH⁺ (Cl=37)).

It was found however that this example could be effectively carried out in a range of solvents at 80° C. and a summary of the results is shown in the following Table 1.

TABLE I
Reaction of 5-Chloro-3-(2-fluoro-4-iodo-phenylamino)-6-methyl-
[1,4]oxazin-2-one with ethyl propiolate at 80° C.,
followed by filtration of product

| | | Loss to | | |
|---|---|---|---|---|
| | Reaction | Liquors at | Isolated | Product Assay |
| Solvent | Time/hours | 0° C./mgmL-1 | Yield/% | w/w % |
| Toluene | 6.5 | 21.57 | 72.1 | 99.6 |
| Acetonitrile | 6.5 | 0.74 | 73.4 | 100 |
| Anisole | 6.5 | 19.85 | 64.5 | 99.7 |
| Chlorobenzene | 6.5 | 30.92 | 60.7 | 100 |
| Isopropanol | 6.5 | 0.47 | 65.6 | 100 |
| n-Butyl acetate | 6.5 | 5.91 | 74.6 | 100 |

### EXAMPLE 4

### 6-Chloro-2-(2-fluoro-4-iodo-phenylamino)-5-methyl-nicotinic acid methyl ester (IIIb)

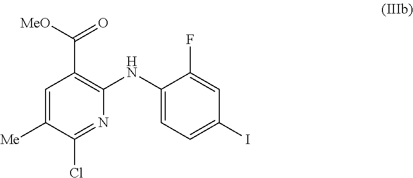

To a stirred slurry of 5-chloro-3-(2-fluoro-4-iodo-phenylamino)-6-methyl-[1,4]oxazin-2-one (2.4 g; 5.8 mmoles) in toluene (24 mL) was added methyl propiolate (1.5 g; 17.5 mmoles; 3.0 equiv). The mixture was then heated to 86° C. After 19 hours, HPLC analysis indicated that the reaction was complete and the mixture was cooled to ambient temperature. The volume of the solution was reduced to ˜⅖ its original volume by vacuum distillation (rotary evaporator), resulting in the crystallisation of some solid. The slurry was allowed to cool to ambient temperature then cooled with ice (external) for 2 hours. The slurry was filtered then washed with fresh toluene (6 mL) and dried, to provide 6-chloro-2-(2-fluoro-4-iodo-phenylamino)-5-methyl-nicotinic acid methyl ester (1.87 g, 76% yield),

δ_{H} (400 MHz, CDCl₃) 2.30 (3H, d, J0.5, ArCH₃), 3.94 (3H, s, COOCH₃), 7.45 (2H, m, ArH), 8.08 (1H, br., J0.5, ArH), 8.39 (1H, ˜t, J8.5, ArH), 10.36 (bs, 1H, NH); m/z (LCMS, ES⁺) 420.9, 422.9 (3:1 MH⁺ (Cl=35):MH⁺ (Cl=37)).

### EXAMPLE 5

### 6-Chloro-5-methyl-2-phenylamino-nicotinic acid ethyl ester (IIIc)

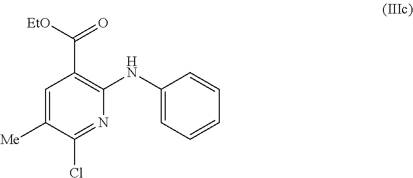

Ethyl propiolate (1.17 mL, 1.13 g, 11.41 mmoles) was charged to a thin slurry of 5-Chloro-6-methyl-3-phenylamino-[1,4]oxazin-2-one 5-Chloro-6-methyl-3-phenylamino-[1,4]oxazin-2-one (2.50 g, 9.51 mmoles) in butyl acetate (14.38 mL, 5.75 rel vols) and the mixture was heated to 120° C. After 21 hours, the reaction was cooled to 0° C. over 2.5 hours and held at 0° C. for 2 hours. The product was then filtered off and washed with butyl acetate (≦5° C., 625.00 μL, 0.25 rel vols) and then dried in a vacuum oven at 45° C., to give 6-Chloro-5-methyl-2-phenylamino-nicotinic acid ethyl ester (1.77 g, 99.0% w/w, 63% yield), m.p. 116-118° C., νₘₐₓ 3245, 1690, 761, 707 cm⁻¹; δ_{H} (400 MHz) 1.41, (3H, t, J7, CH₂CH₃), 2.27 (3H, s, CH₃), 4.37 (2H, q, J7, CH₂CH₃), 7.05 (1H, ˜t, J8, Ar—H), 7.33 (2H, ˜t, J8, Ar—H₂), 7.70 (2H, ˜d, J8, Ar—H₂), 8.04 (1H, s, H−1), 10.14 (1H, s, NH); m/z (HRMS, ES⁺) [MH]⁺ (C₁₅H₁₆ClN₂O₂)=291.0895: Found 291.0896.

### EXAMPLE 6

### 6-Chloro-2-(2-fluoro-phenylamino)-5-methyl-nicotinic acid ethyl ester (IIId)

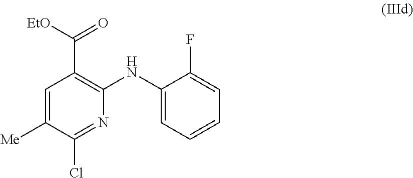

Ethyl propiolate (3.86 mL, 3.74 g, 37.70 mmoles) was charged to a thin slurry of 5-Chloro-3-(2-fluoro-phenylamino)-6-methyl-[1,4]oxazin-2-one (8.00 g, 31.42 mmoles) in butyl acetate (46.00 mL, 5.75 rel vols) and the mixture was heated to 120° C. After 21 hours, the reaction was cooled to 0° C. over 2.5 hours and held at 0° C. for 2 hours. The product was then filtered off and washed with butyl acetate (≦5° C., 2.00 mL, 0.25 rel vols) and then dried in a vacuum oven at 45° C., to give 6-Chloro-2-(2-fluoro-phenylamino)-5-methyl-nicotinic acid ethyl ester (8.31 g, 99.7% w/w, 85% yield), m.p. 122-126° C., νₘₐₓ 3269, 1692, 758 cm⁻¹; δ_{H} (400 MHz) 1.41 (3H, t, J7, CH₂CH₃), 2.32 (3H, d, J1, CH₃), 4.46 (2H, q, J7, CH₂CH₃), 6.96 (1H, m, Ar—H), 7.12 (1H, ddd, Ar—H), 7.15 (1H, m, Ar—H), 8.08 (1H, br.q, J1, Pyr-H), 8.60 (1H, ˜td, J8, 2, Ar—H), 10.36 (1H, s, NH); m/z (HRMS, ES⁺) [MH]⁺ (C₁₅H₁₅ClFN₂O₂)=309.0801: Found 309.0816.

### EXAMPLE 7

### 6-Chloro-2-(4-iodo-phenylamino)-5-methyl-nicotinic acid ethyl ester (IIIe)

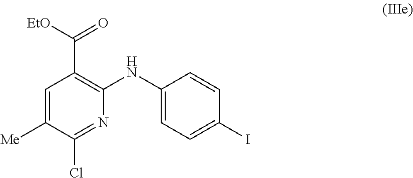

Ethyl propiolate (3.39 mL, 3.28 g, 33.10 mmoles) was charged to a thin slurry of 5-Chloro-3-(4-iodo-phenylamino)-6-methyl-[1,4]oxazin-2-one (10.00 g, 27.58 mmoles) in butyl acetate (57.50 mL, 5.75 rel vols) and the mixture was heated to 120° C. After 21 hours, the mixture was cooled to 0° C. over 2.5 hours and held at 0° C. for 2 hours. The product was then filtered off and washed with butyl acetate (≦5° C., 2.50 mL, 0.25 rel vols) and then dried in a vacuum oven at 45° C., to give 6-Chloro-2-(4-iodo-phenylamino)-5-methyl-nicotinic acid ethyl ester (11.49 g, 100% w/w, 89% yield), m.p. 144-146° C., νₘₐₓ 3249, 1684, 791 cm⁻¹; δ_{H} (400 MHz) 1.41 (3H, t, J7, CH₂CH₃), 1.53 (3H, s, CH₃), 4.38 (2H, q, J7, CH₂CH₃), 7.50 (2H, ˜d, J9, Ar—H₂), 7.60 (2H, ˜d, J9, Ar—H₂), 8.08 (1H, s, H−1), 10.17 (1H, s, NH); m/z (HRMS, ES⁺) [MH]⁺ (C₁₅H₁₅ClIN₂O₂)=416.9861: Found 416.9875.

### EXAMPLE 8

### 5-Chloro-3-(2-fluoro-4-iodo-phenylamino)-6-methyl-[1,4]oxazin-2-one

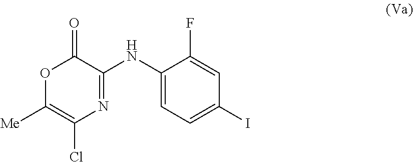

### Method 1—from 3,5-dichloro-6-methyl-[1,4]oxazin-2-one in THF with BF₃

A slurry of 3,5-dichloro-6-methyl-[1,4]oxazin-2-one (50 g, 272.2 mmoles) and 2-Fluoro-4-iodoaniline (73 g; 1.11 equiv) in tetrahydrofuran (1.0 L) was agitated for 10 min at 20° C., then warmed to 40° C. Boron Trifluoride-tetrahydrofuran Complex (57.2 g; 409 mmoles) was added and the temperature was increased to 66° C. over 20 min. After approximately 39 h, the reaction was complete according to HPLC analysis and the mixture was cooled to 20° C. over 1 h. Water (1.0 L; 55.5 moles) was added over ˜3.5 h, during which time the product crystallized. After ˜1 h the slurry was filtered, the filter cake was washed with 1:1 THF:water (100 mL), then dried in a vacuum oven at 45° C. over ˜16 h, to provide 5-chloro-3-(2-fluoro-4-iodo-phenylamino)-6-methyl-[1,4]oxazin-2-one, as a pale brown solid, 83.9 g (80% yield, 99.2% w/w), δ_{H} (400 MHz, CDCl₃), 2.31 (3H, s, CCH₃), 7.48 (1H, dd, J10, 2, ArH), 7.51 (1H, m, ArH), 8.09 (1H, bs, NH), 8.29 (1H, ˜t, J8.5, ArH); m/z (LCMS, APCI) 380.9298 (MH⁺ (Cl=35)).

### Method 2—from 3,5-dichloro-6-methyl-[1,4]oxazin-2-one in chlorobenzene with MsOH

A slurry of DCMO (2.5 g; 13.47 mmoles), 2-fluoro-4-iodoaniline (3.58 g; 1.1 equiv; 14.8 mmoles) and methanesulfonic Acid (1.95 g, 20.2 mmoles) in chlorobenzene (50 mL) was heated at 75° C. for approximately 11 h, when HPLC analysis indicated that there was <5% DCMO remaining. The mixture was allowed to cool to 61° C., then Water (25 mL; 1.39 moles) was added cautiously added over 5 min. The resultant bi-phasic mixture was stirred for ˜10 min. at ˜60° C. and the aqueous phase was removed. The organic phase was washed with water (35 mL) at ˜60° C., then isopropyl alcohol (34 mL]) was added over ˜30 min, maintaining the temperature at 60-66° C. The solution was allowed to cool, over 3 h, from ˜66° C. to ˜40° C., during which time crystallisation occurred. The stirred slurry was then held at ambient for a further 16 h at ambient, then cooled to ˜1° C. for ˜8 h, before being filtered. The filter cake was washed with 1:1 IPA:Chlorobenzene (13 mL) then dried in a vacuum oven at (40° C.), to provide the 5-chloro-3-(2-fluoro-4-iodo-phenylamino)-6-methyl-[1,4]oxazin-2-one (3.21 g, 100% w/w, 63% yield).

### Method 3—from Iatonitrile

### Step 1—Preparation of 3,5-Dichloro-6-methyl-[1,4]oxazin-2-one (DCMO)

To a stirred slurry of triethylamine hydrochloride (24.2 Kg) in toluene (140 Kg) at ambient temperature was added oxallyl chloride (134 Kg) over ˜10 min, during which time the temperature was allowed to rise from 15-30° C. The mixture was then heated to between 70-75° C. and a solution of lactonitrile (48 Kg, 1.0 equiv.) in toluene (22 Kg) was added over 5-6 h, during which time gas was evolved. The mixture was then stirred at 70-75° C. for a further 4 h, after which time lactonitrile had been consumed (<1% by GC). The temperature of the mixture was then reduced and water (250 Kg) was added slowly keeping the temperature below 30° C. The aqueous phase was then removed and the organic phase was filtered through vitacel (ca. 5 Kg), washed with water (250 Kg), then partially distilled to remove water and provide a toluene solution of 3,5-dichloro-6-methyl-[1,4]oxazin-2-one (approx 220 Kg total, 22-26 w/w % DCMO).

### Step 2—5-Chloro-3-(2-fluoro-4-iodo-phenylamino)-6-methyl-[1,4]oxazin-2-one

The DCMO solution in toluene (approx. 220 Kg), as prepared above, was combined with 2-fluoro-4-iodoaniline (77.5 Kg, 1.1 equiv. [based on HPLC assay of the DCMO solution from Step 1) and agitated at ambient. Methane sulfonic acid (36 Kg, 1.25 eqiv.) was added over 30 min and the mixture was then heated at 80-82° C. and held at that temperature for ˜12 h, until HPLC indicated that the concentration of DCMO was <1%. The mixture was then cooled to 20-30° C., followed by the slow addition of methanol (320 Kg), keeping the temperature below 30° C. The temperature of the mixture was then lowered to 10-15° C. and held for 1 h, before filtering under vacuum. The filter cake was washed with methanol (2×60 Kg) then dried at 40-50° C./50 mbar, until loss on drying was <0.5%, to provide 5-chloro-3-(2-fluoro-4-iodo-phenylamino)-6-methyl-[1,4]oxazin-2-one (87 Kg, 64% yield over 2 steps).

### EXAMPLE 9

### 5-Chloro-6-methyl-3-phenylamino-[1,4]oxazin-2-one (Va-II)

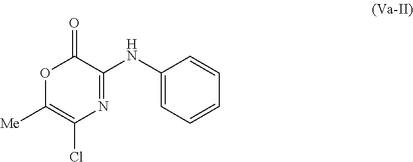

To a solution of 3,5-dichloro-6-methyl-[1,4]oxazin-2-one (8 g, 43.25 mmoles) and aniline (4.52 g, 47.57 mmoles) in tetrahydrofuran (160 mL), at 40° C., boron trifluoride-tetrahydrofuran complex (9.08 g, 64.87 mmoles) was added. The resultant mixture was heated to 68° C. for 48 hours. It was then cooled to 20° C. and held at ambient for 48 h. Water (160 mL) was then added over 3.6 hours. The resulting slurry was held for 1 hour before being filtered and washed with tetrahydrofuran:water (20 mL, 1:1). The solid was dried in a vacuum oven at 40° C. to give 5-Chloro-6-methyl-3-phenylamino-[1,4]oxazin-2-one (4.07 g, 90% w/w, 36% yield), m.p. 133-137° C., νₘₐₓ 3329, 1733, 1057, 754, 687 cm⁻¹; δ_{H} (400 MHz) 2.21 (3H, s, CH₃), 7.08 (1H, ˜t, J8, Ar—H), 7.34 (2H, ˜t, J8, Ar—H₂), 7.91 (2H, ˜d, J8, Ar—H₂), 9.82 (1H, s, NH); m/z (HRMS, ES⁺) [MH]⁺ C₁₁H₁₀ClN₂O₂=237.0425: Found 237.0414.

### EXAMPLE 10

### 5-Chloro-3-(2-fluoro-phenylamino)-6-methyl-[1,4]oxazin-2-one (Va-III)

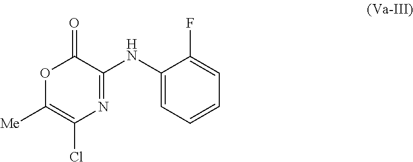

To a solution of 3,5-dichloro-6-methyl-[1,4]oxazin-2-one (10.00 g, 54.45 mmoles) and 2-fluoroaniline (6.79 g, 59.89 mmoles) in tetrahydrofuran (200 mL), under nitrogen, at 40° C., boron trifluoride-tetrahydrofuran complex (11.43 g, 81.67 mmoles) was added. The resultant mixture was heated at 68° C. for 45 hours. It was then cooled to 20° C. over 1 hour and water (200 mL) was added over 4 hours. The resulting slurry was then held for 1 hour before being filtered and washed with tetrahydrofuran:water (1:1). The solid was dried in a vacuum oven at 40° C. to give 5-Chloro-3-(2-fluoro-phenylamino)-6-methyl-[1,4]oxazin-2-one (9.01 g, 99% w/w, 64% yield), m.p. 126-129° C.; νₘₐₓ 3394, 3362, 1735, 1062, 764 cm⁻¹; δ_{H} (400 MHz) 2.31 (3H, s, CH₃), 7.12 (3H, m, 3×Ar Ar—H), 8.15 (1H, br. s, NH), 8.52 (1H, ˜td, J8, 2, Ar—H); m/z (HRMS, ES⁺) [MH]⁺ (C₁₁H₉ClFN₂O₂)=255.0331: Found 255.0327.

### EXAMPLE 11

### 5-Chloro-3-(4-iodo-phenylamino)-6-methyl-[1,4]oxazin-2-one (Va-IV)

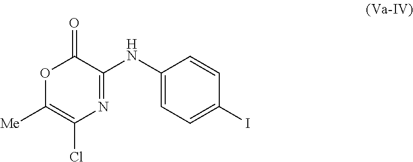

To a solution of 3,5-dichloro-6-methyl-[1,4]oxazin-2-one (8.00 g, 43.25 mmoles) and 4-iodoaniline (10.63 g, 47.57 mmoles) in tetrahydrofuran (160 mL), at 40° C., boron trifluoride-tetrahydrofuran complex (9.08 g, 64.87 mmoles) was added. The resultant mixture was heated at 68° C. for 45 hours. It was then cooled to 20° C. over 1 hour and water (160 mL) was added over 3.6 hours. The resulting slurry was then held for 1 hour before being filtered and washed with tetrahydrofuran:water (1:1, 20 mL). The solid was dried in a vacuum oven at 40° C. to give 5-Chloro-3-(4-iodo-phenylamino)-6-methyl-[1,4]oxazin-2-one (11.6 g, 92% w/w, 67% yield), m.p. 203-205° C. νₘₐₓ 3323, 1725, 1059 cm⁻¹; δ_{H} (400 MHz) 2.21 (3H, s, CH₃), 7.67 (2H, ˜d, J9, Ar—H₂), 7.75 (2H, ˜d, J9, Ar—H₂), 9.95 (1H, s, NH); m/z (HRMS, ES⁺) [MH]⁺ (C₁₁H₉ClIN₂O₂)=362.9392: Found 362.9403.

### EXAMPLE 12

### 5-Chloro-6-methyl-3-(4-nitro-phenylamino)-[1,4]oxazin-2-one (Va-V)

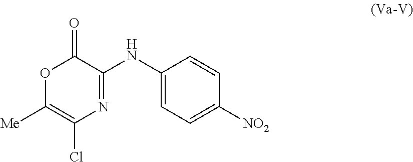

To a solution of 3,5-dichloro-6-methyl-[1,4]oxazin-2-one (2.00 g, 10.81 mmoles) and 4-nitroaniline (1.68 g, 11.89 mmoles) in tetrahydrofuran (40.00 mL), at 40° C., boron trifluoride-tetrahydrofuran complex (2.27 g, 16.22 mmoles) was added. The resultant mixture was heated at 68° C. for 48 hours. It was then cooled to 20° C. over 1 hour and water (40.00 mL) was added over 3.6 hours. The resulting slurry was then held for 1 hour before being filtered and washed with tetrahydrofuran:water (1:1, 20 mL). The solid was dried in a vacuum oven at 40° C. to give 5-Chloro-6-methyl-3-(4-nitro-phenylamino)-[1,4]oxazin-2-one (2.75 g, 99% w/w, 89% yield), m.p. 230-234° C.; νₘₐₓ 3322, 1736, 1566, 1272 cm⁻¹; δ_{H} (400 MHz) 2.24 (3H, s, CH₃), 8.16 (2H, ˜d, Ar—H₂), 8.24 (2H, ˜d, Ar—H₂), 10.43 (1H, s, NH); m/z (HRMS, ES⁺) [MH]⁺ (C₁₁H₉ClN₃O₄)=282.0276: Found 282.0280.

1. A process for preparing a compound of formula (I) 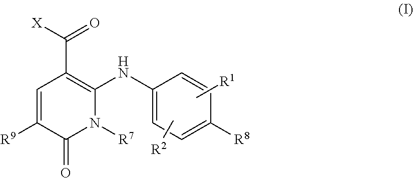
or a pharmaceutically acceptable salt thereof;
where
R⁷ is methyl or ethyl, either of which are optionally substituted with one or more fluorine atoms,
R¹, R², R⁸ and R⁹ are independently hydrogen, hydroxy, halogen, cyano, nitro, trifluoromethyl, difluoromethyl, fluoromethyl, fluoromethoxy, difluoromethoxy, trifluoromethoxy, azido, —SR²¹, —OR²³, —C(O)R²³, —C(O)OR²³, —NR²⁴C(O)OR²⁶, —OC(O)R²³, —NR²⁴SO₂R²⁶, —SO₂NR²³R²⁴, —NR²⁴C(O)R²³, —C(O)NR²³R²⁴, —NR²⁵C(O)NR²³R²⁴, —NR²⁵C(NCN)NR²³R²⁴, —NR²³R²⁴, C₁₋₁₀ alkyl, C₂₋₁₀ alkenyl, C₂₋₁₀ alkynyl, C₃₋₁₀ cycloalkyl, C₃₋₁₀ cycloalkylalkyl, —S(O)ⱼC₁₋₆alkyl, —S(O)ⱼ(CR²⁴R²⁵)ₘ-aryl, aryl, arylalkyl, heteroaryl, heteroarylalkyl, heterocyclyl, heterocyclylalkyl, —O(CR²⁴R²⁵)ₘaryl, —NR²⁴(CR²⁴R²⁵)ₘ-aryl, —O(CR²⁴R²⁵)ₘ-heteroaryl, —NR²⁴(CR²⁴R²⁵)ₘ-heteroaryl, —O(CR²⁴R²⁵)ₘ-heterocyclyl or —NR²⁴(CR²⁴R²⁵)ₘ-heterocyclyl, wherein any of said alkyl, alkenyl, alkynyl, cycloalkyl, aryl, arylalkyl, heteroaryl, heteroarylalkyl, heterocyclyl and heterocyclylalkyl portions are optionally substituted with one or more groups independently selected from oxo (with the proviso that it is not substituted on an aryl or heteroaryl), halogen, cyano, nitro, trifluoromethyl, difluoromethoxy, trifluoromethoxy, azido, —NR²⁴SO₂R²⁶, —SO₂NR²³R²⁴, —C(O)R²³, —C(O)OR²³, —OC(O)R²³, —NR²⁴C(O)OR²⁶, —NR²⁴C(O)R²³, —C(O)NR²³R²⁴, —NR²³R²⁴, —NR²⁵C(O)NR²³R²⁴, NR²⁵C(NCN)NR²³R²⁴, —OR²³, aryl, heteroaryl, arylalkyl, heteroarylalkyl, heterocyclyl, and heterocyclylalkyl, and wherein said aryl, heteroaryl, arylalkyl, heteroarylalkyl, heterocyclyl or heterocyclylalkyl rings may be further substituted with one or more groups selected from halogen, hydroxyl, cyano, nitro, azido, fluoromethyl, difluoromethyl, trifluoromethyl, C₁₋₄alkyl, C₂₋₄ alkenyl, C₂₋₄alkynyl, C₃₋₆ cycloalkyl, C₃₋₆heterocycloalkyl, NR²³R²⁴ and OR²³;
where R²³ is hydrogen, trifluoromethyl, C₁₋₁₀alkyl, C₂₋₁₀alkenyl, C₂₋₁₀alkynyl, C₃₋₁₀cycloalkyl, C₃₋₁₀cycloalkylalkyl, aryl, arylalkyl, heteroaryl, heteroarylalkyl, heterocyclyl, heterocyclylalkyl, phosphate or an amino acid residue, wherein any of said alkyl, alkenyl, alkynyl, cycloalkyl, aryl, arylalkyl, heteroaryl, heteroarylalkyl, heterocyclyl and heterocyclylalkyl portions are optionally substituted with one or more groups independently selected from oxo (with the proviso that it is not substituted on an aryl or heteroaryl), halogen, cyano, nitro, trifluoromethyl, difluoromethoxy, trifluoromethoxy, azido, —NR²¹SO₂R²⁹, —SO₂NR²¹R²⁸, —C(O)R²¹, C(O)OR²¹, —OC(O)R²¹, —NR²¹C(O)OR²⁹, —NR²¹C(O)R²⁸, —C(O)NR²¹R²⁸, —SR²¹, —S(O)R²⁹, —SO₂R²⁹, —NR²¹R²⁸, —NR²¹C(O)NR²⁸R³⁰, —NR²¹C(NCN)NR²⁸R³⁰, —OR²¹, aryl, heteroaryl, arylalkyl, heteroarylalkyl, heterocyclyl, and heterocyclylalkyl, or R²³ and R²⁴ together with the atom to which they are attached form a 4 to 10 membered carbocyclic, heteroaryl or heterocyclic ring, wherein any of said carbocyclic, heteroaryl or heterocyclic rings are optionally substituted with one or more groups independently selected from halogen, cyano, nitro, trifluoromethyl, difluoromethoxy, trifluoromethoxy, azido, —NR²¹SO₂R²⁹, —SO₂NR²¹R²⁸, —C(O)R²¹, —C(O)OR²¹, —OC(O)R²¹, —NR²¹C(O)OR²⁹, —NR²¹C(O)R²⁸, —C(O)NR²¹R²⁸, —SR²¹, —S(O)R²⁹, —SO₂R²⁹, —NR²¹R²⁸, —NR²¹C(O)NR²⁸R³⁰, —NR²¹R³⁰, —OR²¹, aryl, heteroaryl, arylalkyl, heteroarylalkyl, heterocyclyl, and heterocyclylalkyl;
R²⁴ and R²⁵ independently are hydrogen or C₁₋₆ alkyl; or
R²⁴ and R²⁵ together with the atom to which they are attached form a 4 to 10 membered carbocyclic, heteroaryl or heterocyclic ring, wherein said alkyl or any of said carbocyclic, heteroaryl and heterocyclic rings are optionally substituted with one or more groups independently selected from halogen, cyano, nitro, trifluoromethyl, difluoromethoxy, trifluoromethoxy, azido, —NR²¹SO₂R²⁹, —SO₂NR²¹R²⁸, —C(O)OR²¹, —OC(O)R²¹, —NR²¹C(O)OR²⁹, —NR²¹C(O)R²⁸, —C(O)NR²¹R²⁸, —SR²¹, —S(O)R²⁹, —SO₂R²⁹, —NR²¹R²⁸, —NR²¹C(O)NR²⁸R³⁰, —NR²¹C(NCN)NR²⁸R³⁰, —OR²¹, aryl, heteroaryl, arylalkyl, heteroarylalkyl, heterocyclyl, and heterocyclylalkyl;
R²⁶ is trifluoromethyl, C₁₋₁₀alkyl, C₃₋₁₀cycloalkyl, aryl, arylalkyl, heteroaryl, heteroarylalkyl, heterocyclyl or heterocyclylalkyl, wherein any of said alkyl, cycloalkyl, aryl, arylalkyl, heteroaryl, heteroarylalkyl, heterocyclyl and heterocyclylalkyl portions are optionally substituted with one or more groups independently selected from oxo (with the proviso that it is not substituted on an aryl or heteroaryl), halogen, cyano, nitro, trifluoromethyl, difluoromethoxy, trifluoromethoxy, azido, —NR²¹SO₂R²⁹, —SO₂NR²¹R²⁸, —C(O)R²¹, C(O)OR²¹, —OC(O)R²¹, —NR²¹C(O)OR²⁹, —NR²¹C(O)R²⁸, —C(O)NR²¹R²⁸, —SR²¹, —S(O)R²⁹, —SO₂R²⁹, —NR²¹R²⁸, —NR²¹C(O)NR²⁸R³⁰, —NR²¹C(NCN)NR²⁸R³⁰, —OR²¹, aryl, heteroaryl, arylalkyl, heteroarylalkyl, heterocyclyl, and heterocyclylalkyl;
R²¹, R²⁸ and R³⁰ independently are hydrogen, lower alkyl, lower alkenyl, aryl and arylalkyl, and R²⁹ is lower alkyl, lower alkenyl, aryl and arylalkyl; or any two of R²¹, R²⁸, R³⁰ or R²⁹ together with the atom to which they are attached form a 4 to 10 membered to carbocyclic, heteroaryl or heterocyclic ring, wherein any of said alkyl, alkenyl, aryl, arylalkyl carbocyclic rings, heteroaryl rings or heterocyclic rings are optionally substituted with one or more groups independently selected from halogen, cyano, nitro, trifluoromethyl, difluoromethoxy, trifluoromethoxy, azido, aryl, heteroaryl, arylalkyl, heteroarylalkyl, heterocyclyl, and heterocyclylalkyl;
is m is 0, 1, 2, 3, 4 or 5; and j is 0, 1 or 2;
X is OR⁶, SR⁶, —NR⁶R⁵, —N(R¹²)OR⁶, —N(R⁵)SO₂R⁶, C₃₋₁₀cycloalkyl, C₁₋₁₀alkyl, aryl, heteroaryl or heterocyclyl;
wherein R⁶ is a group as defined above for R²³;
R⁵ and R¹² are groups as defined above for a group R²⁴; or
R¹² is linked to R⁶ so as to form a protected derivative thereof;
which method comprises hydrolysis of a compound of formula (II) 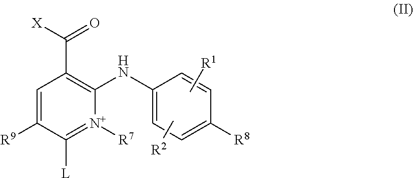
where X, R¹, R², R⁷, R⁸ and R⁹ are as defined above, and L is a leaving group.

2. A process for preparing a compound of formula (II) as defined in claim 1, which process comprises reacting a compound of formula (III) 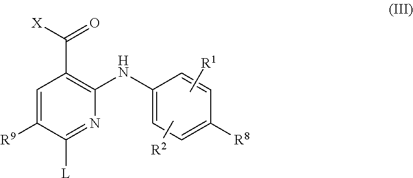
where L, X, R¹, R², R⁶, R⁸ and R⁹ are as defined in claim 1;
with a compound of formula (IV)
R⁷-L¹ (IV)
where R⁷ is as defined in claim 1 and L¹ is a leaving group.

3. A process for preparing a compound of formula (III) as defined in claim 2 which process comprises reacting a compound of formula (V) 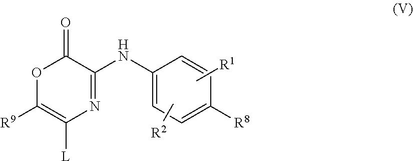
where L, R¹, R², R⁸ and R⁹ are as defined in claim 1;
with a compound of formula (VI) 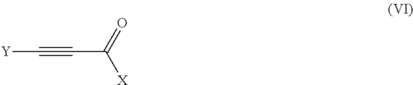
where X is as defined in claim 1, Y is hydrogen or a removable group.

4. A process for preparing a compound of formula (III) as defined in claim 2 which process comprises the steps of (i) reacting a compound of formula (V) 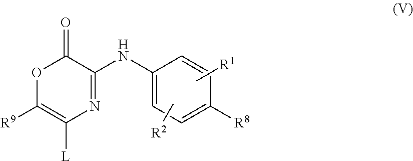
where L, R¹, R², R⁸ and R⁹ are as defined in claim 1;
with a compound of formula (VIa) 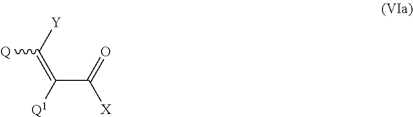
where X is as defined in claim 1, Y is hydrogen or a removable group and Q and Q¹ are independently selected from hydrogen or a group which is readily removeable by to elimination and one of Q or Q¹ is a group which is readily removeable by elimination; and (ii) converting the product of step (i) to a compound of formula (III).

5. A process for preparing a compound of formula (III) as defined in claim 2 which process comprises the steps of (i) reacting a compound of formula (V) 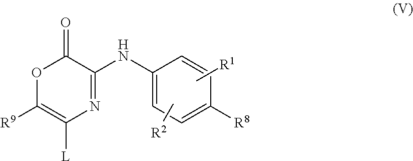
where L, R¹, R², R⁸ and R⁹ are as defined in claim 1;
with a compound of formula (VIa) 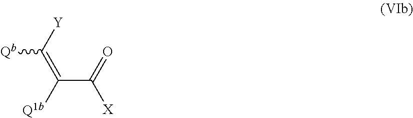
where Y, Q^{b} and Q^{1b} are all hydrogen, to form a dihydropyridine and (ii) oxidising the dihydropyridine to form a pyridine of formula (III).

6. A process for preparing a compound of formula (V) as defined in claim 3 wherein the compound of formula (V) is prepared by reacting a compound of formula (VII) 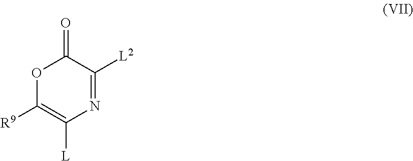
where R⁹ is as defined in claim 1 and L² is a leaving group, with a compound of formula (VIII) 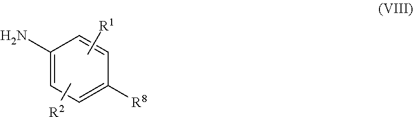
where R¹, R² and R⁸ are as defined in claim 1.

7. A process according to claim 6 wherein the compound of formula (VII) is prepared in situ by reacting a compound of formula (XI) with a compound of formula (X) 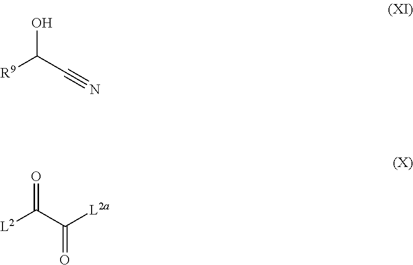
where R⁹ is as defined in claim 1, and L² and L^{2a} are both leaving groups.

8. A process according to any one of claims 1 to 6 wherein R¹ and R² are independently selected hydrogen, halogen, C₁₋₆alkyl, OCH₃ or SCH₃.

9. A process according to any one of claims 1 to 5 wherein X is OR⁶, NHR⁶, —N(R¹²)OR⁶, SR⁶ or CH₂R⁶, where R⁶ and R¹² is selected from hydrogen, or C₁₋₁₀alkyl optionally substituted by hydroxy or cycloalkyl.

10. A process according to any one of claims 1 to 6 wherein R⁸ is hydrogen, fluorine, chlorine, bromine, iodine C₁₋₄alkyl, OCH₃ or SCH₃.

11. A process according to any one of claims 1 to 7 wherein R⁹ is hydrogen, CN, halogen, or C₁₋₄alkyl optionally substituted by one or more groups independently selected from F or CN.

12. A compound of formula (II) as defined in claim 1.

13. A compound of formula (III) as defined in claim 2.

14. A compound of formula (V) as defined in claim 3.

15. A compound according to any one of claims 12 to 14 wherein R⁸ is iodine.

16. A compound according to any one of claims 12 to 14 where R¹ is fluorine.
